(19) United States
(12) Patent Application Publication
(10) Pub. No.: US 2011/0053889 A1
(43) Pub. Date: Mar. 3, 2011

(54) Prenatal and postnatal screening and treatment of critical monosaccharide deficiencies for neurologic and immunologic function

(76) Inventor: Joanne Szabo, Little Rock, AR (US)

(21) Appl. No.: 12/231,383

(22) Filed: Sep. 2, 2008

(51) Int. Cl. A61K 31/70; A61K 31/7004; A61K 31/198; A61K 31/7012; A61P 3/02; A61P 25/00; G06F 19/00; G06F 17/18; G06N 5/02

(52) U.S. Cl. 514/62; 514/23; 514/561; 702/19; 706/52

(57) ABSTRACT

A method for determining the effect of critical glyconutrient dietary supplements in premature and term infants on growth, health, and brain function during early childhood development is described. The method comprises determining a critical target nutrient profile in typical samples of preterm and mother's milk, developing a supplement of the missing critical target nutrients to simulate mother's milk, performing an evaluation of early child development by correlating physiologic brain function with cognitive/behavioral brain function in low birth weight premature and term infants, assessing family influence and home environment on developmental outcome on infants under treatment, and comparing term siblings of low birth weight premature vs. term babies. A composition having the critical nutrients, an algorithm for screening and treatment, and a useful kit for employing the above are also described.

This invention was made with government support under CRIS 6251-51000-002-03S awarded by USDA. The government has certain rights in the invention.

### BACKGROUND OF THE INVENTION

1. Field of the Invention

The present invention generally concerns the role of dietary sugars in development of infants.

The present invention particularly concerns determining monosaccharide composition of human milk oligosaccharides, which offer benefits with regard to neurologic and immunologic function in preterm and term infants. It also concerns development of a glyconutrient supplement designed to enhance developmental outcomes for infants and children, including those who are low birth weight, premature and high-risk.

2. Description of Related Art

Of U.S. Pat. No. 4,115,590 total U.S. live births in 2004, 8.1% (˜333,350) were low birth weight (LBW) infants weighing less than 2500 g. The percentage of infants born at less than 2,500 g has increased 16% since 1990, and the LBW rate in 2004 is the highest level reported since 1969 (Martin et al., 2006). In 2000, the mortality rate for LBW infants was 7.6/1000 live births, with a mortality rate 2× higher in black infants (lyasu et al., 2002). Although recent medical and technological advances have led to improved survival rates, this has been associated with an increased risk of adverse developmental outcomes among these infants. In a population-based study that examined the incidence of school-age disabilities based on birth weight (Avchen et al., 2001), LBW children accounted for 8.5% of all children with specific learning disabilities, 9% of children with emotional handicap, and 18% of children who were educable mentally handicapped. This has resulted in a substantial economic impact of LBW preterm birth both during and following initial hospitalization, even among non-disabled survivors (Petrou et al., 2001).

The importance of early nutrition for normal growth and development is well recognized. This nourishment is a constant feature of the intrauterine environment and is continued following birth in the form of breast milk or formula feeding. Infants born prematurely may be disadvantaged in terms of later growth, immunologic function, and, to a great extent, brain development, since they have not received the same duration, amounts and/or array of in utero nutrients provided to term babies. Many LBW premature infants have increased vulnerability to a variety of diseases and greater likelihood of long-term physiologic and cognitive developmental disorders. The need for adequate postnatal nutrition is, therefore, especially urgent in these infants.

Mother's breast milk (MM), relative to infant formula (F), provides unique advantages to preterm and term infants with regard to cognitive development, resistance to infection, and improved health. Human milk oligosaccharides (HMOs), absent in infant formulas, contain monosaccharides (MS) that are reported to be important in cell interaction and communication.

Breast Milk as Infant Nutrition

The proportion of mothers who begin breast-feeding has been increasing to almost 70% in recent years (Kaufman, 2002), with 64% of all American mothers initiating breastfeeding while in the hospital (45% African American). However, only 29% of all mothers (19% African American) are still breastfeeding at 6 months (reported by Ross Labs, 1998). Likewise, other studies have confirmed a dramatically lower breast-feeding rate among mothers who were Black, poor, young, and less educated (Raisler et al., 1999). Unfortunately, infants who are at highest risk from suboptimal nutrition are those who remain less likely to be breast fed.

Breast milk protects against infections including acute and prolonged diarrhea, respiratory tract infections, otitis media, urinary tract infection, neonatal septicemia, and necrotizing enterocolitis (Hanson, 1998, Barlow, 1976; Moriartey et al., 1979; Winikoff, 1982; Buescher, 1994). In very low birth weight infants fed human milk, the incidence of any infection [human milk (29.3%) vs. formula (47.2%)] and sepsis/meningitis [human milk (19.5%) vs. formula (32.6%)] were significantly reduced compared with exclusively formula-fed very low birth weight infants (Hylander et al., 1998). Likewise, in breast milk-fed infants the incidence of nosocomial sepsis in the intensive care nursery in the first 10 days of life was 5%, during days 11-24 was 9%, and during days 25-38 was 0%, compared with 10%, 20% and 15% in formula fed infants at these respective time periods (El-Mohandes et al., 1997). Breastfeeding affects the expression of certain bacterial virulence factors, resulting in decreased virulence (Howie et al., 1990). The glycoproteins from human milk, which are potential receptor analogues for certain bacteria, prevent microbial adhesion to the epithelial cell surface (Schwertmann et al., 1999).

Breast milk has been shown to have a beneficial effect on cognitive development. Babies who were fed breast milk had an 8 point advantage in mean score on the Bayley Mental Developmental Index over infants of mothers who did not breast feed (Morley et al., 1988). In preterm and term infants, breast milk was associated with higher developmental scores at 18 months as well as significantly higher IQ at 7.5 to 8 years, compared to infants who received no maternal milk (Lucas et al., 1992, 1994, 1996; Florey et al., 1995). Cognitive performance, particularly language-based skills, was especially vulnerable to suboptimal early nutrition. These effects were most notable in premature male infants (Lucas et al., 1998).

Breast milk consumption, not the process of breast feeding, confers the advantages of mother's milk in children born prematurely (Lucas et al., 1992). There was a dose-response relation between the proportion of mother's milk in the diet and subsequent IQ, with greater benefit in preterm children (Lucas et al., 1992, 1994, 1996; Florey et al., 1995; Anderson et al., 1999). Lanting et al. (1994) showed a small advantageous effect of breastfeeding on neurological status at 9 years. Recently, beneficial effects of early breast-feeding have been found to extend through adulthood. Increased duration of breast-feeding, particularly for infants fed ≧8 months, was associated with consistent and statistically significant increases in intelligence, reading, mathematic, and scholastic ability during young adulthood (Horwood and Fergusson, 1998). These results have been confirmed in a recent study (Mortensen et al., 2002) which found these results to be long-lasting, with a significant positive association between duration of breastfeeding (up to 9 months) and adult intelligence, independent of a wide range of possible confounding factors. These studies suggest that breast milk provides critical components, especially to the preterm infant, which are dose related, long-lasting, and may affect early neurologic development to influence later cognitive and behavioral outcomes.

Breast Milk Oligosaccharides

Human milk contains structurally diverse complex oligosaccharides in significant amounts. They form the third major solute after lactose (glucose+galactose) and lipids, and they are present in greater quantities overall than protein (McVeagh and Brand-Miller, 1997). Human milk oligosaccharides are synthesized in the mammary gland through the action of several galactosyltransferases, glucosaminyltransferases, fucosyltransferases, and sialyltransferases, which add specific monosaccharides to “core” oligosaccharides (Egge et al. 1983). To date, more than 130 acidic and neutral oligosaccharides have been isolated. Oligosaccharides are classified as either acidic or neutral according to the presence or absence of sialic acid, or as nitrogen-containing or not, depending on the presence or absence of N-acetylglucosamine. With few exceptions, all oligosaccharides isolated so far contain lactose at their reducing end, with further variations of these core structures dependent on the attachment of fucose and/or N-acetylneuraminic acid residues at different positions of the core region and core elongation chain (McVeagh and Brand-Miller, 1997; Brand-Miller et al., 1994; Kunz and Rudloff, 1993).

Brand-Miller et al. (1994) suggest two competing hypotheses for the biological role of human milk oligosaccharides. In the antiinfectious hypothesis, the oligosaccharides must resist digestion in the small bowel to reach their target organs intact. In the brain development hypothesis, sialic acid needs to be cleaved from the terminal end of acid oligosaccharides and absorbed in the gut. Both hypotheses may be true, because three different families of enzymes are required to completely hydrolyze the oligosaccharides. If only sialidases are present, the sialic acid residue will be cleaved from the terminal position of all of the acidic oligosaccharides, while leaving the rest of the oligosaccharide intact. In this way, sialic acid is made available to the blood while the rest of the oligosaccharides reach the colon (Brand-Miller et al., 1994).

Prevention of Infection.

Epidemiologic findings show that breast fed infants have a lower incidence of bacterial infections within the respiratory, gastrointestinal, and urogenital tracts than do non-breast fed infants (Newburg, 1999). In vitro studies have shown that human milk oligosaccharides inhibit the attachment of bacteria to the respiratory, gastrointestinal, and urinary tracts (Howie et al., 1990; Brand-Miller et al., 1994).

Respiratory tract infections and acute otitis media are important causes of morbidity in children, and H. influenzae and S. pneumonthniae are the major bacterial agents associated with such infections. Human milk inhibits the attachment of H. influenzae and S. pneumonthniae to human pharyngeal or buccal epithelial cells in vitro, presumably by acting as a receptor analogue (Andersson et al., 1986). In rabbits, oligosaccharides block the adherence of bacteria to epithelial cells in vitro, and have been shown to attenuate the course of pneumococcal pneumonia and bacteremia when given intratracheally. In infant rats, neoglycoconjugates of the active oligosaccharides prevented colonization of the nasopharynx when administered intranasally (Idanpaan-Heikkila et al., 1997). Human milk oligosaccharides are soluble receptor analogues of epithelial cell-surface carbohydrates, and compete with epithelial ligands for pathogenic bacteria and viruses. They protect the infant from infection by preventing pathogens from adhering to human cells by binding to proteins on the pathogen (adhesins, lectins or hemaglutinnins) and binding to enterotoxin receptors.

Human milk oligosaccharides (HMOs) appear to serve as soluble ligands that prevent pathogenic microorganisms from adhering to and invading the epithelia of the gastrointestinal and urogenital tracts. HMOs resist digestion in the small intestine of most breast fed infants and undergo fermentation in the colon (Brand-Miller et al., 1998). Although intact HMOs may be absorbed, the majority is delivered to the colon apparently intact where they provide the main source of carbon and energy for intestinal bacterial metabolism. Because colonic bacteria express a wide range of enzymes, including fucosidases and sialidases (Engfer et al., 2000), substantial degradation of human milk oligosaccharides occurs in the colon. As a result, only small amounts of intact HMOs are found in the feces of term and preterm breast fed infants (Sabharwal et al., 1991).

Human milk oligosaccharides also protect the breast fed infant from infection by promoting the growth of bifidobacteria. By the end of the first week of life, Bifidobacteria represent 95% of the total bacterial population in the feces of exclusively breast fed infants, whereas in formula fed infants they form less than 70%. Exogenous Bifidobacteria-supplementation of newborn rats resulted in intestinal colonization, with a significant reduction in the incidence of NEC, compared with controls and E. coli-treated animals (Caplan et al., 1999).

The growth promoting factors for L. bifidus var. pennsylvanicus have been isolated from the wheys of human milk and colostrum, as well as the proteolytic digests of human casein. The glycopeptide fraction isolated from the soluble portion of human milk casein digests stimulated the growth of L. bifidus var. pennsylvanicus to the same extent as a whey glycopolypeptide fraction, and it contained between 60 and 70% carbohydrate consisting of galactose, galactosamine, fucose, glucosamine, and sialic acid (Beskorovainy et al. 1979).

HMOs containing D-glucosamine or N-acetylglucosamine (the bifidus factor) are necessary for the growth of Bifidobacteria species (Gyorgy et al., 1974; Kunz and Rudloff, 1993). Such oligosaccharides form precursors in the biosynthesis of muramic acid, a component of the bacterial cell wall. Bifidobacteria decrease the intestinal pH by producing lactic acid that is not well absorbed, and these innocuous organisms use nutrients at the expense of those required for the growth of other pathogenic microorganisms. These factors inhibit the proliferation of many pathogenic microorganisms such as Shigella sp., E. coli, Streptococcus faecalis and Clostridium, which become predominant after weaning from breast milk (McVeagh and Brand-Miller, 1997; Kunz and Rudloff, 1993).

In preterm newborn infants, gangliosides (acidic glycosphingolipids) supplementation at concentrations similar to human milk resulted in significantly lower fecal E. coli content than infants fed with control milk formula within the first week of life, and higher fecal Bifidobacterial counts by 1 month of life (Rueda et al., 1998). Data suggest that sialylated oligosaccharides and other compounds with conjugated sialylated glycoproteins and glycolipids may function as receptor-analogous structures for bacterial adhesins. These compounds may modify the intestinal microflora in the neonate to reduce the infectious capacity of these bacteria. Bifidobacteria growth in the intestine may also be enhanced by sialylated oligosaccharides (Sabharwal et al., 1991) and fortification of infant formula with N-acetylneuraminic acid-containing substances (Idota et al., 1994).

Enhancement of Neurologic Development.

Glycoprotein-carbohydrate in brain tissue consists of approximately 65% acidic sialoglycopeptides, which contain galactose, fucose, N-acetylglucosamine, mannose, and N-acetylneuraminic acid. Approximately 25% of the glycoprotein-carbohydrate of brain tissue is associated with mannose-rich oligosaccharide polymers (Brunngraber et al., 1976).

During animal and human development, ganglioside concentration increases approximately three-fold within a short period. In rat cerebrum, this occurs from birth to the 17^{th} postnatal day (largest increase by 11^{th} to 13^{th} day). In human cerebral cortex, ganglioside concentrations increase from the 15^{th} fetal week to about 6 months postnatal age (largest increase by term). The relative increase of gangliosides during this period was more rapid than that of phospholipids, and the changes in brain ganglioside pattern that were characteristic of the developmental stages of the rat were equally pronounced in the human brain (Vanier et al., 1971).

In the rat, postnatal brain development is associated with large increases in the concentration of brain glycoproteins. During the first month of life, greatest changes occur in the levels of glycoprotein galactose, glucosamine and mannose, which usually occupy “inner” positions in the oligosaccharide chains of glycoproteins with smaller increases in fucose and sialic acid, which are located exclusively at terminal positions on the oligosaccharides. The relatively greater increase in glucosamine, mannose and galactose (as compared to fucose and sialic acid) in brain glycoproteins during development is due to the preferential synthesis in older animals of a population of glycoproteins with oligosaccharide chains consisting largely of “core” sugars, and containing only a small proportion of fucose and sialic acid (Margolis et al., 1976).

In rats, there was a 50% increase in the glycopeptide content during the first four weeks to the adult level. Adult rat brain was composed of two glycopeptide groups. The neutral glycopeptides accounted for about 45% of the glycopeptide carbohydrate and contained mostly N-acetylglucosamine and mannose. The acidic glycopeptides accounted for about 55% of the glycopeptide carbohydrate and contained galactose, N-acetylgalactosamine, N-acetylglucosamine, and mannose as well as fucose and N-acetylneuraminic acid (Krusius et al., 1974).

In the human brain, the developmental pattern (4^{th} month of intrauterine life to the 30^{th} year of life) of the soluble and insoluble fractions of glycoproteins was examined to correlate with structural and functional modifications occurring during CNS development. Developmental changes occurred earlier in the soluble glycoprotein fraction when compared with the insoluble fraction. The protein bound sialic acid of the soluble fraction did not change during intrauterine life, but underwent a rapid decrease at birth. The protein-bound sialic acid content of the insoluble glycoproteins was stable up to the 30^{th} day of life, and increased after this age to stabilize at adult levels. The protein-bound fucose of the soluble fraction decreased more slowly after birth than sialic acid, while a continued increase of protein-bound fucose was found in the insoluble glycoproteins (Federico and Di Benedetta, 1978).

Human milk oligosaccharides are metabolized in the term and preterm infant to provide these important monosaccharide substrates, although the relative efficiency in preterm infants is still not well understood. In the premature infant, hepatic and renal function, which are important in monosaccharide metabolism, are immature. Although it remains to be elucidated, it is suspected that enzymatic function in premature infants may be inadequate to efficiently metabolize the lactose in infant formulas to these important glycoprotein substrates. Therefore, it appears that monosaccharide supplementation of infant formula would prove beneficial to infant CNS development during the critical early periods of brain growth, between the third trimester and 2 years post-term (Dobbing, 1981).

Important Breast Milk Monosaccharides

Monosaccharides form an essential part of the “language” enabling healthy cells to interact and communicate. There are eight monosaccharides commonly found in the structures coating studied cells: glucose (Glc), galactose (Gal), fucose (Fuc), N-acetylglucosamine (GlcNAc), N-acetylneuraminic acid (NeuNAc, “sialic acid”), N-acetylgalactosamine (GalNAc), xylose (Xyl), and mannose (Man) (Murray, 1996). The monomers of human milk oligosaccharides (HMO) are D-glucose, D-galactose, L-fucose, N-acetylglucosamine, and N-acetylneuraminic acid. Inventor's preliminary studies have shown that the monosaccharides fucose, glucosamine, mannose, and sialic acid, while present in human milk, are not found in significant/equal amounts in standard term and premature infant formulas. As shown in FIG. 1 (Zubay, 1993), these monosaccharides may serve as precursors to important components of breast milk oligosaccharides that are involved in the immunologic and cognitive benefits observed in breast fed infants.

Although many of these monosaccharides can be derived from glucose (FIG. 1), individual studies of these sugars show that there may be separate transport systems and that certain sugars may be utilized preferentially over glucose if they are available.

Several of these sugars are similar to the eight monosaccharides commonly found in the structures coating cells, and may form an essential part of the “language” enabling healthy cells to interact and communicate.

The monosaccharide components of human milk oligosaccharides that appear particularly promising, in terms of importance for cognitive and immunological function, include fucose, glucosamine, mannose, and sialic acid. Therefore, Inventor includes a brief summary of known biological activities of these monosaccharides and their precursors. Inventor then discusses in greater detail what is known regarding the metabolism and excretion of these sugars.

Biological Activities

Fucose.

Fucose plays an important role in cell-to-cell communication and occurs in abundance in glycoproteins and glycolipids (Flowers, 1981; Babbar et al., 1990). Fucose is present in the glycoprotein and glycolipid red blood cell antigens which are important in determination of blood type, and fucose inhibits macrophage- and neutrophil-chemotactic factors, which are important in the immune system cascade (Flowers, 1981). Fucose is distributed in macrophages (Tsukada and Spicer, 1988), and stimulates rabbit macrophage migration (Takata et al., 1987). A small neutral fucosyloligosaccharide from human milk may act as an analogue receptor in the intestinal mucosa to protect suckling mice from the diarrheagenic effects of heat-stabile enterotoxin of E. coli. (Newburg et al., 1990).

Fucose glycoproteins are an important component of brain cells in animals (Brunngraber et al., 1975; Webster and Klingman, 1980) and humans (Brunngraber et al., 1975). Fucosylated glycoproteins are involved in synaptic adhesion and connectivity, and they are components of several neurotransmitter receptors. Approximately 85% of synaptic plasma membrane (SPM) glycoproteins contain fucose. SPM proteins were assessed in brains of developing offspring from rats fed a protein-deficient diet during lactation (Druse et al., 1982). Neonatal undernutrition significantly reduced brain weights and quantitatively affected neuronal and synaptic development, with a 33-50% deficiency of synaptic plasma membrane (SPM) proteins in the brains of undernourished offspring. A significant finding of this study was the rehabilitative component in which brain weights recovered (to 91% of control weights), and earlier synaptic effects were reversed when nutritional rehabilitation through free access to an adequate diet of standard laboratory chow began on the 21^{st} postnatal day.

A rare inherited disorder of fucose metabolism, leukocyte adhesion deficiency type II (LAD II), leads to immunodeficiency caused by the absence of carbohydrate-based selectin ligands on the surface of neutrophils, as well as severe mental and psychomotor retardation. In in vitro studies, the fucosylation defect of LAD II fibroblasts could be corrected by addition of L-fucose to the culture medium. In humans, oral supplementation of fucose in a patient with LAD II resulted in clinical disappearance of infections and fever, return to normal neutrophil counts, and improved psychomotor capabilities (Marquardt et al., 1999). Thus, provision of a simple monosaccharide can alleviate major symptoms caused by an as yet undefined genetic defect in fucose metabolism.

N-Acetylglucosamine (GlcNAc).

N-acetylglucosamine also occurs in abundance in glycoproteins and glycolipids in animals and humans. Glucosamine is essential to various biological activities, including immunologic and neurologic function. Glucosamine has been shown to have antiviral activity in mice inoculated with the human influenza virus (F loch and Werner, 1976) and in rabbits inoculated with herpesvirus eye infections (Perez et al., 1989). GlcNAc inhibits production of radicals by neutrophils as well as release of neutrophil-derived elastase, which limit tissue damage and associated inflammation (Kamel et al., 1991). GlcNAc glycoproteins are also found in the microsomal and synaptosomal tissue fractions of chick and rat brain, suggesting a role in nerve function (Webster and Klingman, 1980; Brunngraber et al., 1975).

Mannose.

Mannose is involved in various biologic activities. It helps to prevent bacterial attachment and infection by competitively binding to bacterial lectins, occupying sites that would normally bind host cell mannose receptors (Beuth et al., 1994). When a topical solution of mannose was applied to maternal vaginas prior to delivery, E. coli bacteremia was significantly reduced from 77% in control to 25% in newborn mice from mannose-treated rats (Cox and Taylor, 1990). Injected mannose also reduced experimentally induced E. coli bacteriuria in rats within 1 day of treatment (Michaels et al., 1983). In rat pups orally infected with E. coli K1, strains expressing mannose-resistant adhesins had significantly higher colonization and invasion rates than non-MR strains (Wullenweber et al., 1993).

Mannose is found in several glycoconjugate components of the immune system, including glycoproteins of human peripheral blood lymphocytes in vitro (Brown et al., 1977). Macrophage receptors bind mannose, which facilitates phagocytosis (Stahl, 1990; 1992). IgG contains a mannose-binding protein (MBP) capable of activating the complement cascade involved in tissue inflammation (Dwek et al., 1995). It is probable that MBP binds to a wide range of microorganisms and may also function as an opsonin (Lipscombe et al., 1992). MBP is present in serum of normal newborn infants at birth, and achieves its highest level within 5 days of birth (Terai and Kobayashi, 1993). In preterm infants, there is a developmental maturation of MBP levels (Lau et al., 1995), with significantly lower levels in preterm compared to term infants and adults. Low serum levels of mannose-binding lectin was associated with an increased risk of acute respiratory tract infections, particularly during the vulnerable period from age 6 through 17 months, when the adaptive immune system is immature (Koch et al., 2001). Another significant risk factor for infection is the occurrence of mutations in genes for mannose binding protein, with about twice the prevalence of mutations in the MBP gene in children with than without infection (Summerfield et al., 1997). Dietary mannose supplementation has also been used to reverse clinical and biochemical symptoms in a patient with carbohydrate-deficient glycoprotein syndrome (CDGS) type Ib (Alton et al., 1997; Niehues et al., 1998; Freeze, 1998).

Mannose was also found in glycoproteins obtained from the microsomal and synaptosomal fractions of rat brain (Brunngraber et al., 1975) and in the glycolipids from chick brain (Webster and Klingman, 1980). Approximately 25% of the glycoprotein-carbohydrate of brain tissue was associated with mannose-rich oligosaccharide polymers (Brunngraber et al., 1976). As previously stated, there are significant increases in glycoprotein mannose levels during the postnatal development of rat brain. Relatively greater increases in brain mannose-containing glycoproteins occur in older animals due to the synthesis of glycoproteins with oligosaccharide chains consisting largely of “core” sugars (Margolis et al., 1976).

N-Acetylneuraminic Acid (Sialic Acid).

Sialic acids is a group of over 36 naturally occurring compounds derived from neuraminic acid. They are present in oligosaccharide chains of mucins, glycoproteins and gangliosides and are important for the function of cell membranes and membrane receptors (McVeagh and Brand-Miller, 1997).

Sialic acid is essential for several important biological activities. Sialic acid glycoconjugates are potential receptor analogues for certain bacteria, and may prevent microbial adhesion to the epithelial cell surface (Schauer, 1982, 1997). Sialic acids also form an integral part of gangliosides which are concentrated in the plasma membranes of nerve cells, especially the region of nerve endings and dendrites. In humans, the highest ganglioside concentrations are found in the cerebral and cerebellar grey matter. The accretion rate is greatest during the period of dendritic arborization and synaptogenesis, which occurs from 25 postconceptional weeks to 4 years postnatally. During this time, the concentration of gangliosides increases approximately three-fold, attaining a maximal value at around 5 years of age. Sialic acid content of brain tissue increases from 0.6 umol/g in early fetuses up to 1.2 umol/g tissue in early childhood (McVeagh and Brand-Miller, 1997). The plasma lipid-bound sialic acid level at birth was somewhat lower in preterm than fullterm infants, but increased more rapidly in preterm infants, such that there were no significant differences between preterm and full-term infants by 5 days of age. Infant levels of plasma lipid-bound sialic acid were significantly lower than adult values, and increased rapidly following birth (Sasaki et al., 1989).

Metabolism and Excretion

Fucose.

Endogenous fucose is produced in the sugar-nucleotide form (GDP-fucose) from GDP-mannose via a dehydratase and an epimerase-reductase enzyme (see FIG. 1). Exogenous fucose is converted to fucose-1-phosphate by fucokinase and then to GDP-fucose by a pyrophosphorylase enzyme (see FIG. 1), and may be directly incorporated into fucose-containing proteins and macromolecules with little or no metabolism to other sugars (Kaufman and Ginsburg, 1968; Wiese et al., 1997). In in vitro animal studies, fucose is absorbed from the small intestine by a non-active diffusion transport process (Bihler, 1969). Cells may also possess a specific facilitative transporter for fucose (Wiese et al., 1994). Oral or injected fucose may be incorporated into glycoproteins (Martin et al., 1998).

Ingested fucose is readily absorbed from the small intestine (Bihler, 1969), and in healthy volunteers oral fucose (50-100 mg/kg body weight) led to peak serum fucose concentrations at 1 hour, with clearance half-time of 100 minutes (Marquardt et al., 1999). Absorbed fucose is extensively incorporated either directly or after metabolism into glycoproteins (Martin et al., 1998; Wiese et al., 1997; Flowers, 1981).

In rats, ingested fucose was shown to be excreted primarily in the urine, but also in feces and expired CO₂ [intestinal microflora are primarily responsible for CO₂ formation (Bocci and Winzler, 1969)]. In adult humans, fucose was eliminated in the urine (Bell and Talukder, 1972), with concentrations of fucose-containing glycopeptides increasing markedly during the latter stages of pregnancy and during lactation (Lemonnier et al., 1978). In human infants, fucose-containing oligosaccharides have been identified in the feces (Whyte et al., 1978).

N-Acetylglucosamine (GlcNAc).

In GlcNAc synthesis, dietary glucosamine is first phosphorylated by glucokinase or hexokinase, and then acetylated by an acetyltransferase enzyme. Then, mutase and pyrophosphorylase enzymes convert GlcNAc into a form that can be metabolized (Martin et al., 1998), with GlcNAc metabolism regulated by insulin (Yki et al., 1998). In rats, GlcNAc is metabolized by liver lysosomes (Kuranda and Aronson, 1985).

Since GlcNAc can be deacetylated, it provides an endogenous source of glucosamine and plays an important role in protein glycosylation (Martin et al., 1998). In rat pups, GlcNAc was important in glycosylation reactions in the developing intestine, with decreased incorporation of GlcNAc into rat pup epithelial cell surface glycoproteins whose mothers were fed a low-protein diet (Babbar et al., 1990).

Dietary GlcNAc provides a more direct source of the sugar for glycosylation. In rats, oral dosing of N-acetylglucosamine resulted in significant absorption of GlcNAc and glucosamine (deacetylated metabolite of GlcNAc). Within 3 hours of oral consumption of radiolabeled GlcNAc, there was considerable radioactivity found in amino acid fractions of rat liver and small intestine (Capps et al., 1966). In humans, ingested GlcNAc was readily absorbed, as seen by a significant rise in serum levels of GlcNAc within 1 hour after oral administration of 1 gram of the sugar (Talent and Gracy, 1996). When glucosamine was ingested, about one-half of the dose was oxidized, with much of the remainder distributed into glycoconjugates (Martin et al., 1998). In animal studies, GlcNAc is primarily eliminated in urine and expired CO₂ (Capps et al., 1966).

Mannose.

Endogenous mannose can be converted from glucose to GDP-mannose, mainly in the liver by kinase, isomerase, mutase, and pyrophosphorylase enzymes of the glycolysis metabolic pathway (see FIG. 1). In animals and humans, ingested mannose is readily absorbed by a mannose-specific, glucose-tolerant transporter that is localized in intestinal epithelial cells (Bihler, 1969; Brydon et al., 1987; Alton et al., 1998). In the guinea-pig, intravenously injected mannose was uniformly distributed throughout the intravascular, extravascular and epithelial compartments of the small intestine (Kingham et al., 1978). In suckling rats, radiolabeled mannose was incorporated into intestinal microvillus membranes (Babbar et al., 1990). Mannose could be directly utilized for glycoprotein biosynthesis (Alton et al., 1998).

Following oral administration in humans, mannose is efficiently absorbed and increases blood mannose levels in a dose-dependent manner with increasing oral doses of mannose (Alton et al., 1997). In humans, exogenous mannose is preferentially utilized for glycoconjugate synthesis (Alton et al., 1998; Berger et al., 1998), and radiolabeled mannose was incorporated to a greater extent than glucose in serum (Berger et al., 1998).

In humans, pharmacokinetic studies show peak blood mannose concentrations approximately 90 minutes following oral ingestion (0.07-0.21 g/kg body weight), and clearance half-time about 4 hours. Blood mannose concentrations rose in a dose-dependent fashion for all participants (Alton et al., 1997). In rats, orally ingested, radiolabeled mannose was rapidly absorbed into the serum within 1 hour and was readily distributed throughout body fluids and tissues, with highest levels found in the liver and intestine. Mannose was extensively incorporated into plasma and tissue glycoproteins, and glycoprotein incorporation in organs increased 2- to 6-fold at 1-8 hours (Alton et al., 1998; Brydon et al., 1987).

Animal studies have shown that there is active renal reabsorption of mannose, with very little excreted in the urine. The luminal surface of the proximal tubules have two different transport sites, with one shared by glucose and galactose and the other specific for mannose (Silverman et al., 1970). Mannose is also capable of crossing the placenta when available at normal physiological concentrations, allowing incorporation of maternal sugar into the fetus. Human amniotic fluid has been shown to contain mannose comparable to maternal blood concentrations (Alton et al., 1998).

N-Acetylneuraminic Acid (Sialic Acid).

Sialic acid metabolism plays an important role in the biosynthesis of glycoconjugates (Schauer, 1982, 1997), and enzymes involved in sialic acid metabolism also regulate metabolism of other essential monosaccharides and glycoconjugates (Revilla et al., 1998). Endogenous sialic acid may be produced from glucosamine by an epimerase enzyme, as demonstrated when a radiolabled glucosamine injection resulted in 25-30% of protein-bound radioactivity in the form of sialic acid (Martin et al., 1998). N-acetylmannosamine may also serve as a sialic acid precursor through the action of kinase and phosphatase enzymes (see FIG. 1).

Sialic acid appears to be readily absorbed when ingested, based on its appearance in multiple glycoconjugates throughout the body (Schauer, 1982, 1997). Free sialic acid concentrations were similar in adults and breast fed infants (both groups significantly greater than formula fed infants), while adult concentrations of bound and total sialic acid in saliva were significantly higher than breast and formula fed infants (Tram et al., 1997).

Sialic acid is found in red blood cell (RBC) membranes (Viverge et al., 1985; 1990), as well as several human body fluids including amniotic fluid, milk, saliva, serum, cerebrospinal fluid, and urine (Hayakawa et al., 1993; Martin, 1998). In humans, urinary excretion of sialyl-oligosaccharides is significantly increased during lactation (Lemonnier et al., 1978), which coincides with markedly increased serum sialic acid levels during late-stage pregnancy (Lemonnier and Bourrillon, 1976).

The preceding summaries of fucose, glucosamine, mannose, and sialic acid biological activities, metabolism and excretion suggest that these monosaccharides: 1) are important for cell-to-cell communication and interaction, 2) are important components of glycoproteins and glycolipids, 3) are important factors for immunologic competence, 4) may be produced endogenously depending upon appropriate substrate and enzyme activity, and 5) may be readily absorbed and utilized when supplied orally.

The preceding background information attests to the importance of mother's milk for overall infant health, including cognitive and neurologic development. This is particularly important in the LBW preterm infant, who has not received the same duration, amounts and/or array of in utero nutrients compared to term babies. While is seems clear that LBW infants would benefit from mother's milk, a large number of mothers of LBW infants choose not to or cannot produce sufficient breast milk to support normal growth and development. Infant formulas do not provide the oligosaccharides found in breast milk, which results in absent or decreased amounts of monosaccharides instrumental in promoting infant health and normal brain development, particularly in high-risk infants. Studies within this invention are unique in that they provide quantitative data on the effects of nutrition on physiologic and cognitive/behavioral brain function during infancy and early childhood development (0-3 yrs.). They provide a clearer understanding of the role of critical monosaccharides found in human milk oligosaccharides, and provide a scientific basis upon which nutritional supplementation of these monosaccharides to infant formula may help to ameliorate or prevent adverse developmental outcomes in high-risk infants. Furthermore, results from this study lead to improved formula composition for term infants as well. The well-documented beneficial effects of breast milk on postnatal neurological development in term (Florey et al., 1995; Morley et al., 1988; Lanting et al., 1994) and preterm infants (Lucas et al., 1992, 1994, 1996) emphasizes the need for prospective research into the basis for these effects. Such research is important not only to be able to maximize the nutritional benefits for these infants—both normal and at-risk—whose postnatal nutrition is formula-based, but also potentially to rehabilitate LBW preterm infants that have been inadequately nourished during the prenatal and postnatal periods.

The immediate and long-term consequences of various diets on developmental parameters involved in growth and body composition, general health, physiological and cognitive/behavioral brain functioning in LBW premature and term infants need to be assessed.

Inventor's review of the literature shows that few of the monosaccharides that comprise important dietary oligosaccharides have been studied extensively in humans or animals. Inventor found no information regarding the bioavailability and dosing of these monosaccharides in infants and children, and limited information in adults and animals.

Although much is known regarding glucose metabolism, placental transport and fetal utilization, little information along these lines is available for the sugars appearing in Inventor's studies described hereinafter. There is little available information regarding maternal levels, transplacental transfer at different stages of gestation, fetal uptake and utilization, and metabolism of these monosaccharides in infants and young children.

Lack of critical monosaccharides for ganglioside incorporation in the preterm infant, either as a result of decreased intrauterine exposure or their lack in preterm breast milk or infant formula, accounts for some of the cognitive and behavioral deficits these infants experience.

### SUMMARY OF THE INVENTION

This invention disclosure presents a series of investigations and examples that accomplish the following specific objectives in creating appropriate diets for low birth weight (LBW) premature and term infants and children.

The primary objective of this invention is to develop a supplement of critical monosaccharides that when added to preterm and term formulas will provide a sound nutritional diet through the first 6 months of life, simulating the composition in mother's milk.

Another object in accordance with the present invention is to perform a longitudinal evaluation (0-3 years) of growth and body composition (anthropometrics, BIA, and DEXA) and evaluate general health, incidence of infection and morbidity using a questionnaire in LBW premature and term infants assigned to mother's milk, formula, or baby formula supplemented with monosaccarides.

Yet another object of this invention is to correlate physiologic brain function (electrophysiologic/behavioral assessments of heart rate, respiratory rate, and EEG recordings in response to paradigms designed to measure social and startle response, language discrimination, and visual attention) with cognitive/behavioral brain function (standardized tests of intelligence, achievement, language, temperament and behavior) in LBW premature and term infants assigned to mother's milk, formula, or baby formula supplemented with monosaccarides during infancy, from birth through 3 years of age.

A further object is to assess the influence of family and home environment (maternal intelligence and psychopathology, and family socioeconomic status, stability, adaptability and cohesion) on developmental outcome of LBW premature and term infants assigned to mother's milk, formula, or baby formula supplemented with monosaccarides to determine the role of environment vs. nutrition on growth, health, physiologic and cognitive/behavioral brain functioning from birth through 3 years.

A further, preferred object is to compare term siblings of the LBW premature vs. term babies at age 3 years to provide preliminary complementary information about calculated estimate of heritability influencing the nutrition-development relationship. This question of heritability is further determined by contemplating a genetic-dietary link of blood group phenotypes and monosaccaride deficiencies and premature birth.

In accordance with the abovementioned objects, this invention contemplates a method for determining the effect on premature and term infants of critical nutrient supplements in their diet on growth, health, physiologic and cognitive/behavioral brain function during early childhood development. This method entails determining the target nutrient content in typical samples of mother's milk (MM) that is either missing, or found at a low level in low birth weight (LBW) preterm newborns. The contemplated method further involves developing a supplement of these critical nutrients (NS) that can be added to preterm and term formulas (F) in order to simulate their composition in mother's milk.

A more specific and preferred embodiment of this invention is a method for determining the effect of critical monosaccharide supplements in the diet of premature and term infants on their growth, health, immunocompetence, physiologic and cognitive/behavioral brain function during early childhood development.

An equally preferred embodiment in accordance with this invention is a method for developing a supplement of these critical monosaccharides (MS) that can be added to preterm and term formulas (F) in order to simulate their composition in mother's milk. This adding of critical monosaccharides to preterm and term infant formulas is to continue through the first 6 months of life. The preferred monosaccharide composition comprises fucose, glucosamine, mannose, and sialic acid. Endogenous sialic acid may be produced from glucosamine by an epimerase enzyme

Another, most preferred embodiment of this invention comprises performing a longitudinal evaluation of early child development upon receiving the diet treatment. This evaluation contemplates determinations of growth and body composition, general health, immunocompetence, incidence of infection, and morbidity, and involves the evaluating for a period of 0-3 years. The growth and body composition is evaluated by anthropometrics, which comprise measurements of weight, length or height, head circumference and the like using standardized techniques. The growth and body composition may be further evaluated by BIA and by DEXA.

Another preferred embodiment is to evaluate the role of monosaccharides as a prebiotic through examination of the development of natural and acquired immunity when formulas are supplemented in LBW premature and term infants. Some analyses include Fluorescent in Situ Hybridization of gut microbiota, and Enzyme-linked Immunospit assay of circulating immunoglobulin-secreting cells, which indirectly indicates gut immunological events, and Toll-like receptor 2.

Another most highly preferred embodiment in accordance with this invention is a method for correlating physiologic brain function with cognitive/behavioral brain function in premature and term infants assigned to MM, F, or MS+F diet groups during infancy, from birth through 3 years of age. Evaluation of physiologic brain function comprises electrophysiologic/behavioral assessments of heart rate, respiratory rate, and EEG recordings in response to paradigms designed to measure social and startle response, language discrimination, and visual attention. Evaluation of the cognitive/behavioral brain function is assessed by standardized tests of intelligence, achievement, language, temperament and behavior.

A further, highly preferred embodiment comprises assessing the influence of family and home environment on the developmental outcome of premature and term infants assigned to MM, F, or MS+F diet groups in order to determine the role of environment versus nutrition on growth, health, physiologic and cognitive/behavioral brain functioning from birth through 3 years of age. The influence of family and home environment assessment comprises a consideration of maternal intelligence and psychopathology, together with family socioeconomic status, stability, adaptability and cohesion.

Another, and most preferred embodiment, is the comparison of term siblings of the LBW premature babies with term babies at 3 years of age, in order to provide preliminary complementary information about heritability influencing the nutrition-development relationship. The comparing evaluates grasp force data, finger-length data, resting state recordings, heart rate analyses, respiratory rate and skin conductance. Herein the grasp force compares left-right hand strength isometric force differences, the finger length compares ratios of second to fourth finger lengths, the resting state is compared based on EEG, EMG and EOG data coupled with quantitative analyses of EEG, autonomic activity and video recordings, and the heart rate analysis comprises measuring heart beat-to-beat intervals in resting periods and in association with stimuli.

Another most preferred embodiment is a composition comprising a custom blend of critical monosaccharides to supplement common commercial term and preterm formulas to simulate their composition in mother's milk. This composition contains the critical monossacharides comprising one or more members of the group comprising fucose, mannose, glucosamine and sialic acid. Endogenous sialic acid may be produced from glucosamine by an epimerase enzyme, if it is active. In this composition, the critical monosaccarides are pharmaceutically compounded in a powder form, in a pharmaceutically acceptable carrier for intravenous administration or enteral feeding.

Yet another highly preferred embodiment is a method for determining a probability of adverse reproductive events in a woman of reproductive age typically in gestation, by determining the presence of one or more biomarkers known to correlate with a known risk of adverse reproductive events. A sample of blood, serum, plasma, mother's milk, saliva or cord blood is analyzed. The biomarkers are fucose, mannose, glutamic acid sialic acid, ABO and Lewis phenotypes. The adverse reproductive events comprise premature birth and glyconutrient deficiencies leading to compromised growth, health, physiologic and cognitive/behavioral brain function in premature and term infants during early childhood development. The correlating is performed by an algorithm that is trained to identify patterns in women with presence or absence of the biomarkers in adverse reproductive events.

Another, highly preferred embodiment is a kit useful for screening for monossacharide deficiencies and defining treatment in LBW premature and term infants. The kit contains devices and reagents and the abovedescribed computer algorithm. The devices are instruments and labware useful in measuring the abovedescribed biomarkers. A manual is also included.

Still further embodiments and advantages of the invention will become apparent to those skilled in the art upon reading the entire disclosure contained herein.

### BRIEF DESCRIPTION OF THE DRAWINGS

FIG. 1: Shows a schematic flow diagram representation of the monosaccharide interconversions.

FIG. 2 shows breast milk values on day 1-6, day 7-10 and 1 month for glucose.

FIG. 3 shows breast milk values on day 1-6, day 7-10 and 1 month for galactose.

FIG. 4 shows breast milk values on day 1-6, day 7-10 and 1 month for fucose.

FIG. 5 shows breast milk values on day 1-6, day 7-10 and 1 month for glucosamine.

FIG. 6 shows breast milk values on day 1-6, day 7-10 and 1 month for mannose.

FIG. 7 shows breast milk values on day 1-6, day 7-10 and 1 month for sialic acid.

FIG. 8 shows breast milk values on day 1-6, day 7-10 and 1 month for and galactosamine.

FIG. 9 shows infant formula monosaccaride levels.

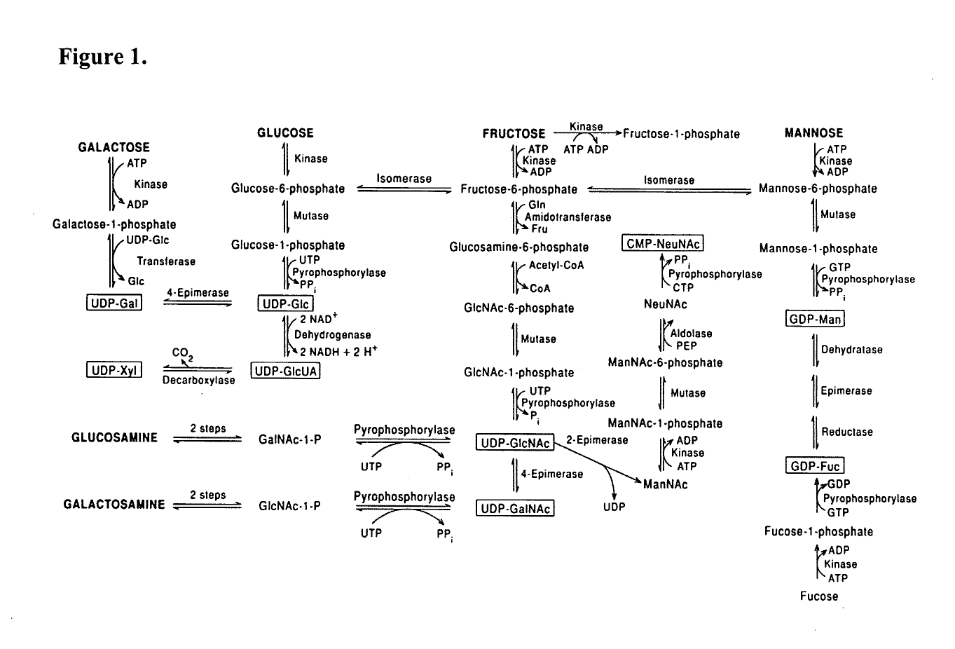

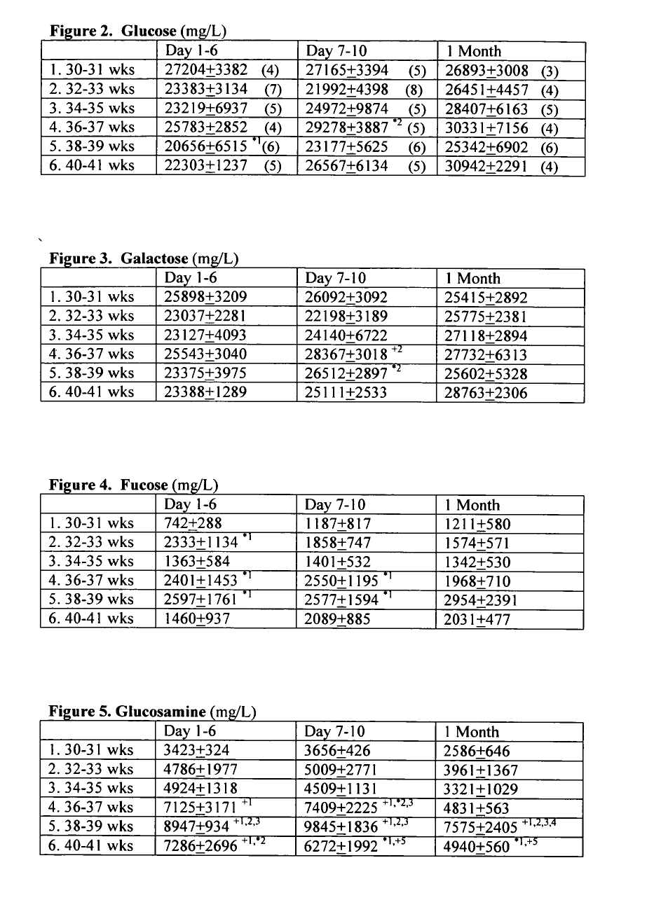

In FIG. 2 and subsequent figures, all values are expressed as M+SD and n=( ). For between group measures, *p<0.05, +p<0.01.

### DESCRIPTION OF THE PREFERRED EMBODIMENTS

### Glyconutrition in Low Birth Weight and Term Infants

### Effects on Growth, Body Composition, Psychophysiologic, and Cognitive/Behavioral Development

### Specific Aim 1

### Example 1

### Analyses of the Monosaccharide Content of Term and Preterm Breast Milk and Infant Formulas

### INTRODUCTION

Samples of hydrolyzed term and preterm breast milk and infant formulas were evaluated for total levels of the following monosaccharides: glucose, galactose, fucose, glucosamine, mannose, sialic acid and galactosamine using standardized techniques for anion-exchange liquid chromatography with integrated pulsed amperometric detection (Eberendu, 2005). The resulting values in term and preterm breast milk and infant formula are listed below in Table 1.

The following monosaccharides are higher in term than in preterm breast milk by the following percentages: glucose 13%, galactose 7%, fucose 37%, and mannose 79%. On the other hand, the following monosaccharides are lower in term than in preterm breast milk by the following percentages: glucosamine 60%, sialic acid 11-20%. Fucose was not detected in term and preterm formulas, nor was glucosamine which serves as a precursor for sialic acid. Mannose content of infant formulas was less than that found in term breast milk. Sialic acid content, obtained from the limited available literature (Wang et al., 2001; Brand-Miller et al., 1994; Sanchez-Diaz et al., 1997), was significantly less in formula compared to term breast milk.

TABLE 1
Monosaccharide Contents of Breast Milk and Infant Formula (mg/ml)

| | Term¹ | Preterm² | Term³ | Preterm³ |
|---|---|---|---|---|
| Sugar | Breast Milk | Breast Milk | Formula | Formula |
| Glucose | 36.587 | 31.950 | 34.180 | 47.332 |
| Galactose | 36.643 | 34.232 | 34.485 | 14.414 |
| Fucose | 2.979 | 1.874 | ND | ND |
| Glucosamine | 1.126 | 1.806 | ND | ND |
| Mannose | .912 | .195 | .753 | .580 |
| Sialic Acid | .816^{a}-.879^{b} | .977^{a} | .233-.266^{c} | — |

¹n = 1,
²n = 6,
³n = 2;
ND = not detected;
— not done
^{a}Wang et al., 2001;
^{b}Brand-Miller et al., 1994;
^{c}Sanchez-Diaz et al., 1997

The estimated daily intake in infants of the monosaccharides (mg/kg) of interest are listed in Table 2 (based on an estimated oral intake of 120 ml/kg/day of breast milk).

The maximum safe total dose of sugars used as dietary supplements can be estimated based on ADME (absorption, distribution, metabolism, excretion) and safety data, and use of a margin-of-safety (Gardiner, 2004). These values, based on daily dose (mg/kg/d) and daily dose for a 68 kg human, are listed in Table 3. Based on available data, it is estimated that all of the glyconutritional sugars daily dose would have to be divided into two to three doses per day to maintain blood levels.

Although excessive doses of some of these sugars can be potentially toxic (e.g., single mannose doses of >200 mg/kg body wt induces osmotic diarrhea and limits the maximal serum concentrations that can be reached by oral uptake), this has no relevance for oral mannose therapy and other monosaccharides, since the uptake capacity of the gut provides natural protection (Alton et al., 1997).

TABLE 2
Estimated daily intake of monosaccharides (mg/kg) in infants

| | Term | Preterm | Term | Preterm |
|---|---|---|---|---|
| Sugar | Breast Milk | Breast Milk | Formula | Formula |
| Glucose | 4,390 | 3,834 | 4,102 | 5,680 |
| Galactose | 4,397 | 4,108 | 4,138 | 1,730 |
| Fucose | 357 | 225 | ND | ND |
| Glucosamine | 135 | 217 | ND | ND |
| Mannose | 109 | 23 | 90 | 70 |
| Sialic Acid | 98-105 | 117 | 28-32 | — |

ND = not detected;
— not done

TABLE 3
Estimated Safe Daily Dose of Glyconutritional Sugars

| | | Daily Dose (mg) |
|---|---|---|
| Glyconutritional Sugar | mg/kg/d | in 68 kg human |
| Glucose | 2,200 | 150,000 |
| Galactose | 735 | 50,000 |
| Fucose | 500 | 34,000 |
| N-acetylglucosamine | 15 | 1,000 |
| Mannose | 779 | 53,000 |
| N-acetylneuraminic acid | 2 | 140 |

### Example 2

### Maternal-Fetal Monosaccharide Levels in Preterm and Term Pregnancies

### INTRODUCTION

Early nutrition is critical for normal fetal and infant growth and development. With regard to fetal nutrition, glucose metabolism, placental transport and fetal utilization have been described, but there is little known regarding other important nutritional components for the fetus. There is increased transplacental transport of many nutrients as pregnancy progresses; however, this may result in relative deficiencies if the infant is delivered prematurely. Although several important sugars are metabolized from glucose, there is little information on additional monosaccharides that may be important for fetal growth and development.

The neonate receives additional exposure to important oligosaccharides postnatally through mother's breast milk. There is little information on the monosaccharides that comprise important dietary oligosaccharides found in human breast milk. Inventor's studies have shown that in addition to glucose and galactose, human milk contains fucose, glucosamine, mannose, sialic acid, and galactosamine. Concentrations of these sugars in human milk vary based on gestational as well as postnatal age. Inventor's studies have shown that premature, term and soy formulas only contain glucose and galactose. Therefore, postnatal diets are likely unable to adequately compensate for intrauterine deficiencies.

There is evidence from animal and human studies that the monosaccharides fucose, glucosamine, mannose, and sialic acid are important in brain development (Brunngraber et al., 1975, 1976; Webster and Klingman, 1980; Druse et al., 1982; McVeagh and Brand-Miller, 1997). A lack of critical monosaccharides for incorporation into brain gangliosides during critical intrauterine and postnatal periods may account for some of the cognitive and behavioral deficits in preterm infants.

Although many monosaccharides can be derived from glucose, individual studies of these sugars show that there may be separate transport systems, and certain sugars may be utilized preferentially over glucose. Infants born prematurely may be disadvantaged in terms of these additional monosaccharides because of 1) less intrauterine exposure (duration, amounts and/or array of nutrients, 2) inadequate postnatal nutrition to compensate for deficiencies, and 3) lack of enzymes to metabolize other monosaccharides from glucose, resulting in limited de novo synthesis.

Purpose of Study

Inventor designed this study to evaluate maternal-fetal levels of several monosaccharides that are incorporated into brain gangliosides and routinely comprise human milk oligosaccharides, but are lacking in infant formulas. This study provides preliminary information regarding early fetal exposure and requirements of these additional monosaccharides.

Methods

Subjects and Design: Inventor studied 19 healthy mother-infant pairs: 11 preterm (30-37 wks gestation) and 8 term (38-42 wks gestation). Mothers ranged in age from 17 to 33 years. Mothers were excluded for significant alcohol, tobacco or drug use; significant medical complications (infection, diabetes, chronic hypertension, pre-eclampsia) or chronic medication use (anti-depressants, etc.). Infants were appropriate weight for gestational age and were excluded for major illness, congenital or surgical anomalies.

For all mothers enrolled into the study, maternal blood was collected within 24 h prior to delivery. Intrauterine fetal exposure to monosaccharides was determined from cord umbilical venous blood obtained from a clamped cord sample immediately after the baby was delivered and before placental separation.

Blood samples were collected and centrifuged, and serum was separated and stored at −20° C. Serum monosaccharide concentrations (glucose, galactose, fucose, glucosamine, mannose, and galactosamine) were determined by HPAEC-PAD. The temperature and acid concentration used for hydrolysis of neutral and amino saccharide-containing complex carbohydrates were detrimental to sialic acids, which were processed and analyzed separately.

### Results

TABLE 4
Monosaccharide Levels in Maternal Serum (mg/L)

| | Fucose | GalNH₂ | GlcNH₂ | Galactose | Glucose | Mannose | Sialic Acid |
|---|---|---|---|---|---|---|---|
| Preterm | 40.0 ± 44.9 | 28.0 ± 10.2 | 795.2 ± 181.9 | 285.6 ± 107.9 | 417.2 ± 185.4 | 227.1 ± 90.5 | 580.7 ± 170.1 |
| Term | 28.0 ± 3.9 | 26.8 ± 7.4 | 748.0 ± 160.7 | 273.3 ± 56.7 | 379.6 ± 139.1 | 228.5 ± 40.6 | 615.0 ± 56.8 |
| p = | 0.462 | 0.778 | 0.566 | 0.772 | 0.636 | 0.968 | 0.593 |
| M ± SD | | | | | | | |

Maternal serum fucose, galactosamine (GalNH₂), glucosamine (GlcNH₂), galactose, glucose, mannose, and sialic acid were not significantly different between mothers delivering preterm or term infants. Inventor's results suggest that maternal serum concentrations of these monosaccharides remain relatively constant between 30 and 42 wks gestation.

TABLE 5
Monosaccharide Levels (mg/L) of Maternal vs. Umbilical Venous
Cord Serum (Preterm)

| | Fucose | GalNH₂ | GlcNH₂ | Galactose | Glucose | Mannose | Sialic Acid |
|---|---|---|---|---|---|---|---|
| Maternal | 40.0 ± 44.9 | 28.0 ± 10.2 | 795.2 ± 181.9 | 285.6 ± 107.9 | 417.2 ± 185.4 | 227.1 ± 90.5 | 580.7 ± 170.1 |
| Cord | 10.4 ± 2.7 | 10.8 ± 2.4 | 395.8 ± 69.6 | 149.8 ± 24.6 | 557.8 ± 173.1 | 99.5 ± 20.8 | 258.6 ± 53.5 |
| p = | 0.041 | 0.000 | 0.000 | 0.001 | 0.081 | 0.000 | 0.000 |
| M ± SD | | | | | | | |

Maternal vs. cord glucose levels were not significantly different for preterm pregnancies. Preterm umbilical venous cord levels were significantly less than maternal levels for all other monosaccharides studied: fucose, galactosamine (GalNH₂), glucosamine (GlcNH₂), galactose, mannose, and sialic acid.

TABLE 6
Monosaccharide Levels (mg/L) of Maternal vs. Umbilical Venous Cord
Serum (Term)

| | Fucose | GalNH₂ | GlcNH₂ | Galactose | Glucose | Mannose | Sialic Acid |
|---|---|---|---|---|---|---|---|
| Maternal | 28.0 ± 3.9 | 26.8 ± 7.4 | 748.0 ± 160.7 | 273.3 ± 56.7 | 379.6 ± 139.1 | 228.5 ± 40.6 | 615.0 ± 56.8 |
| Cord | 9.4 ± 2.3 | 8.7 ± 0.9 | 361.0 ± 44.1 | 132.4 ± 14.5 | 407.4 ± 90.3 | 83.0 ± 13.3 | 112.9 ± 102.4 |
| p = | 0.000 | 0.000 | 0.000 | 0.000 | 0.643 | 0.000 | 0.000 |
| M ± SD | | | | | | | |

Maternal vs. cord glucose levels were not significantly different for term pregnancies. Term umbilical venous cord levels were significantly less than maternal levels for all other monosaccharides studied: fucose, galactosamine (GalNH₂), glucosamine (GlcNH₂), galactose, mannose, and sialic acid.

TABLE 7
Monosaccharide Levels of Umbilical Venous Cord Serum (mg/L)

| | Fucose | GalNH₂ | GlcNH₂ | Galactose | Glucose | Mannose | Sialic Acid |
|---|---|---|---|---|---|---|---|
| Preterm | 10.4 ± 2.7 | 10.8 ± 2.4 | 395.8 ± 69.6 | 149.8 ± 24.6 | 557.8 ± 173.1 | 99.5 ± 20.8 | 258.6 ± 53.5 |
| Term | 9.4 ± 2.3 | 8.7 ± 0.9 | 361.0 ± 44.1 | 132.4 ± 14.5 | 407.4 ± 90.3 | 83.0 ± 13.3 | 112.9 ± 102.4 |
| p = | 0.411 | 0.030 | 0.232 | 0.092 | 0.039 | 0.067 | 0.001 |
| M ± SD | | | | | | | |

Cord umbilical venous serum levels of galactosamine (GalNH₂), glucose, and sialic acid were significantly higher in preterm fetuses.

TABLE 8
Monosaccharide Content of Cord vs. Maternal Serum (%)

| | Fucose | GalNH₂ | GlcNH₂ | Galactose | Glucose | Mannose | Sialic acid |
|---|---|---|---|---|---|---|---|
| Preterm | 41.1 ± 14.6 | 36.3 ± 10.0 | 49.8 ± 11.9 | 50.0 ± 12.4 | 208.8 ± 285.7 | 42.7 ± 15.2 | 44.4 ± 10.5 |
| Term | 34.7 ± 11.6 | 35.7 ± 14.5 | 51.9 ± 19.8 | 51.6 ± 17.8 | 123.4 ± 55.8 | 38.3 ± 13.6 | 19.4 ± 18.7 |
| p | 0.313 | 0.922 | 0.797 | 0.824 | 0.378 | 0.527 | 0.007 |
| M ± SD | | | | | | | |

Inventor evaluated the percentage of monosaccharide content of umbilical cord vs. maternal serum. The percentage of sialic acid was significantly higher in the preterm fetus, while levels of the other monosaccharides were not significantly different.

### DISCUSSION

Placental transport of various substances is complex and influenced by the following:

specific receptor proteins

hydrolysis in the placenta

synthesis in the placenta

fetal regulation of transport across the placenta

rate of fetal metabolism may affect cord blood levels.

Placental transport capacity increases with advancing gestation, probably due to an increased number of transporter proteins as surface area increases (Hay, 1995). Glucose and galactose are transported by carrier-mediated and diffusional processes (Quraishi and Illsley, 1999). Fucose has a specific facilitative transporter (Wiese et al., 1994). Concentrations of fucose-containing glycopeptides increase markedly during the latter stages of pregnancy and during lactation (Lemonnier et al., 1978). Mannose is capable of crossing the placenta when available at normal physiological concentrations, allowing incorporation of maternal sugar into the fetus. Mannose concentrations in human amniotic fluid are comparable to maternal blood concentrations (Alton et al., 1998). Sialic acid is found in red blood cell membranes (Viverge et al., 1985; 1990), as well as amniotic fluid, milk, serum, and urine (Hayakawa et al., 1993; Martin et al., 1998). In humans, urinary excretion of sialyl-oligosaccharides significantly increases during lactation (Lemonnier et al., 1978), which coincides with markedly increased serum sialic acid levels during late-stage pregnancy (Lemonnier and Bourrillon, 1976).

Hereinabove results describe maternal and fetal concentrations at preterm and term gestation, and indicate monosaccharide concentrations to which the fetus is exposed. Inventor's studies were not designed to determine uterine and fetal uptake and metabolism, and results likely indicate the sum of uteroplacental-fetal transfer, synthesis and degradation.

### CONCLUSIONS

Maternal serum levels of fucose, galactosamine, glucosamine, galactose, glucose, mannose and sialic acid did not change significantly as pregnancy progressed towards term.

Glucose levels between mother and fetus were not significantly different in preterm or term pregnancies. This indicates effective glucose transport to the fetus throughout gestation.

Other monosaccharides found in fetal serum included fucose, galactosamine, glucosamine, galactose, mannose, and sialic acid, although in amounts significantly less than maternal levels in both preterm and term pregnancies.

Cord serum levels of galactosamine, glucose, and sialic acid were significantly higher in preterm fetuses. This suggests that preterm infants were exposed to higher concentrations of these monosaccharides in utero, implying a greater need for these substrates during critical periods of early development.

Higher monosaccharide content of cord vs. maternal serum (%) sialic acid in the preterm fetus, while concentrations of other monosaccharides were not different, suggests a significantly greater need for sialic acid during critical periods of intrauterine development.

These findings suggest that for the premature infant there is potentially a greater nutritional need for these monosaccharides:
if they are not obtained in utero,
if they cannot be metabolized from glucose,
if exogenous dietary sugars are unavailable in the newborn infant's diet.

### Example 3

### Maternal Lewis Phenotype is Associated with Human Milk Monosaccharide Content

### INTRODUCTION

Human milk oligosaccharides (HMOs), unique to breast milk, vary in content in term (T) vs. preterm (P) milks. Previous studies have shown that HMO content is related to mother's ABO and Lewis blood-group phenotypes. Inventor has found, in a related study, that P delivery is significantly increased among mothers with Lewis recessive phenotype.

Purpose of Study

Human milk oligosaccharides (HMOs) are unique to breast milk, and their content varies between mothers and between term and preterm milk. The oligosaccharide spectrum and content of mother's milk is genetically determined and related to her ABO and Lewis secretor status (Viverge et al., 1985, 1990; Thurl et al., 1997; Nakhla et al., 1999; Erney et al., 2001). The Lewis secretor is expressed by oligosaccharides rich in fucose, N-acetylglucosamine and sialic acid (Viverge et al., 1990; D'Adamo and Kelly, 2001). Several oligosaccharides are missing in the milk of non-secretor or Lewis-negative individuals (Kobata, 2004), which suggests that breastfeeding by a secretor or nonsecretor mother might offer different degrees of immunologic protection to the infant (LePendu, 2004).

The present Example examined the hypothesis that maternal Lewis blood-group phenotype is associated with monosaccharide composition of human breast milk in preterm and term infants.

Methods

Inventor enrolled 35 breast-feeding mother-infant pairs: 23 preterm (30-37 wks gestation) and 12 term (≧38 wks gestation). Mothers were excluded if there were significant alcohol, tobacco or drug use; significant medical complications (infection, diabetes, chronic hypertension, pre-eclampsia); or chronic medication use (anti-depressants, etc.). Infants were excluded if there were major illness, congenital or surgical anomalies. Mothers were classified according to the following Lewis blood-group phenotypes: secretor (a−b+), non-secretor (a+b−), or recessive (a−b−).

Mothers expressed and collected their initial breast milk samples at 1 to 6 days following delivery, immediately before feeding the infant. Individual samples were frozen as soon after collection as possible, and stored until monosaccharide analyses were performed using standardized techniques for anion exchange chromatography (Mannatech, Coppell, Tex.). Samples of hydrolyzed term and preterm breast milk were evaluated for levels of the following monosaccharides: glucose, galactose, fucose, glucosamine, mannose, and sialic acid.

Results

Inventor compared breast milk monosaccharides at days 1-6 for preterm and term infants based on Lewis blood-group phenotypes as listed in Tables 9-11. All results are M±SD.

### Breast Milk Monosaccharides (d. 1-6)

TABLE 9
Combined Lewis Secretor (a−, b+)/Non-secretor Types (a+/−, b−)

| (mg/L) | Preterm (n = 20) | Term (n = 11) | P = |
|---|---|---|---|
| Glucose | 24714 ± 4117 | 21404 ± 4751 | 0.05 |
| Galactose | 24254 ± 3111 | 23381 ± 2927 | 0.45 |
| Fucose | 1838 ± 1179 | 2080 ± 1501 | 0.62 |
| Glucosamine | 5125 ± 2314 | 8192 ± 2024 | 0.001 |
| Mannose | 115 ± 96 | 78 ± 105 | 0.34 |
| Sialic Acid | 983 ± 263 | 749 ± 200 | 0.02 |
| Galactosamine | 238 ± 130 | 345 ± 82 | 0.02 |

Glucose and sialic acid levels were significantly higher in preterm breast milk, while glucosamine and galactosamine were significantly lower.

TABLE 10
Lewis Secretor (a−, b+)

| (mg/L) | Preterm (n = 7) | Term (n = 6) | P = |
|---|---|---|---|
| Glucose | 24173 ± 3907 | 19562 ± 5601 | 0.11 |
| Galactose | 24165 ± 3369 | 22290 ± 3162 | 0.33 |
| Fucose | 3122 ± 1020 | 2760 ± 1738 | 0.65 |
| Glucosamine | 6548 ± 2744 | 8650 ± 1718 | 0.13 |
| Mannose | 126 ± 88 | 78 ± 138 | 0.46 |
| Sialic Acid | 1008 ± 311 | 658 ± 154 | 0.03 |
| Galactosamine | 348 ± 169 | 366 ± 72 | 0.82 |

In Lewis secretor mothers, sialic acid was significantly higher in preterm breast milk.

TABLE 11
Lewis Non-Secretor Types (a+/−, b−)

| (mg/L) | Preterm (n = 13) | Term (n = 5) | P = |
|---|---|---|---|
| Glucose | 25006 ± 4352 | 23616 ± 2451 | 0.52 |
| Galactose | 24302 ± 3105 | 24690 ± 2234 | 0.80 |
| Fucose | 1146 ± 448 | 1265 ± 578 | 0.65 |
| Glucosamine | 4360 ± 1702 | 7642 ± 2420 | 0.005 |
| Mannose | 108 ± 102 | 78 ± 63 | 0.55 |
| Sialic Acid | 969 ± 247 | 860 ± 207 | 0.39 |
| Galactosamine | 179 ± 41 | 319 ± 94 | 0.000 |

In Lewis non-secretor type (a+/−, b−) mothers, glucosamine and galactosamine were significantly lower in preterm breast milk.

In order to determine if breast milk monosaccharide content for preterm infants at term adjusted age was similar to term infants, Inventor analyzed breast milk monosaccharide content based on secretor types in Tables 12-14.

Glucose and galactose levels were significantly higher in preterm breast milk; and glucosamine, sialic acid, and galactosamine were significantly lower. In Lewis secretor mothers, glucosamine and galactosamine were significantly lower in preterm breast milk.

Breast Milk Monosaccharides (Term Adjusted Age)

TABLE 12
Combined Lewis Secretor (a−, b+)/Non-secretor Types (a+/−, b−)

| (mg/L) | Preterm (n = 12) | Term (n = 11) | P = |
|---|---|---|---|
| Glucose | 28718 ± 5650 | 21404 ± 4751 | 0.003 |
| Galactose | 27044 ± 4424 | 23381 ± 2927 | 0.03 |
| Fucose | 1777 ± 1008 | 2080 ± 1501 | 0.57 |
| Glucosamine | 4318 ± 2204 | 8192 ± 2024 | 0.000 |
| Mannose | 131 ± 141 | 78 ± 105 | 0.32 |
| Sialic Acid | 514 ± 176 | 749 ± 200 | 0.007 |
| Galactosamine | 152 ± 65 | 345 ± 82 | 0.000 |

TABLE 13
Lewis Secretor (a−, b+)

| (mg/L) | Preterm (n = 4) | Term (n = 6) | P = |
|---|---|---|---|
| Glucose | 27251 ± 6902 | 19562 ± 5601 | 0.09 |
| Galactose | 26540 ± 5707 | 22291 ± 3162 | 0.16 |
| Fucose | 2783 ± 774 | 2760 ± 1738 | 0.98 |
| Glucosamine | 5223 ± 2380 | 8650 ± 1718 | 0.03 |
| Mannose | 92 ± 45 | 78 ± 138 | 0.85 |
| Sialic Acid | 537 ± 128 | 658 ± 154 | 0.23 |
| Galactosamine | 196 ± 88 | 366 ± 72 | 0.01 |

In Lewis non-secretor type mothers, glucose was significantly higher in preterm breast milk; glucosamine, sialic acid, and galactosamine were significantly lower.

TABLE 14
Lewis Non-Secretor Types (a+/−, b−)

| (mg/L) | Preterm (n = 8) | Term (n = 5) | P = |
|---|---|---|---|
| Glucose | 29452 ± 5283 | 23616 ± 2451 | 0.04 |
| Galactose | 27296 ± 4071 | 24690 ± 2234 | 0.22 |
| Fucose | 1274 ± 687 | 1265 ± 578 | 0.98 |
| Glucosamine | 3865 ± 2121 | 7642 ± 2420 | 0.01 |
| Mannose | 151 ± 171 | 78 ± 63 | 0.39 |
| Sialic Acid | 503 ± 203 | 860 ± 207 | 0.01 |
| Galactosamine | 129 ± 40 | 319 ± 94 | 0.000 |

Inventor next analyzed preterm breast milk monosaccharide content according to secretor vs. non-secretor types, as shown in Tables 15-16.

Preterm Breast Milk

TABLE 15
Breast Milk Monosaccharides (d. 1-6)

| | Non-secretor (n = 13) | Secretor (n = 7) | |
|---|---|---|---|
| (mg/L) | (a+/−, b−) | (a−, b+) | P = |
| Glucose | 25006 ± 4352 | 24173 ± 3907 | 0.68 |
| Galactose | 24302 ± 3105 | 24165 ± 3369 | 0.93 |
| Fucose | 1146 ± 448 | 3122 ± 1020 | 0.000 |
| Glucosamine | 4360 ± 1702 | 6548 ± 2744 | 0.04 |
| Mannose | 108 ± 102 | 126 ± 88 | 0.70 |
| Sialic Acid | 969 ± 247 | 1008 ± 311 | 0.76 |
| Galactosamine | 179 ± 41 | 348 ± 169 | 0.003 |

At d 1 to 6, fucose, glucosamine, and galactosamine levels were significantly less in breast milk of non-secretor type mothers compared to secretor mothers.

TABLE 16
Breast Milk Monosaccharides (Term Adjusted Age)

| | Non-secretor (n = 8) | Secretor (n = 4) | |
|---|---|---|---|
| (mg/L) | (a+/−, b−) | (a−, b+) | P = |
| Glucose | 29452 ± 5283 | 27251 ± 6902 | 0.55 |
| Galactose | 27296 ± 4071 | 26540 ± 5707 | 0.80 |
| Fucose | 1274 ± 687 | 2783 ± 774 | 0.006 |
| Glucosamine | 3865 ± 2121 | 5223 ± 2380 | 0.34 |
| Mannose | 151 ± 171 | 92 ± 45 | 0.52 |
| Sialic Acid | 503 ± 203 | 537 ± 128 | 0.77 |
| Galactosamine | 129 ± 40 | 196 ± 88 | 0.09 |

At term adjusted age, fucose levels remained significantly less in breast milk of Lewis non-secretor type mothers compared to secretor mothers.

### CONCLUSION

The following changes are noted in preterm and term adjusted age milk (vs. term).

| | Glucose | Galactose | Glucosamine | Sialic Acid | Galactosamine |
|---|---|---|---|---|---|
| Birth | | | | | |
| Lewis Combined | ↑ | | ↓ | ↑ | ↓ |
| Secretor | | | | ↑ | |
| Non-secretor types | | | ↓ | | ↓ |
| Term Adjusted | | | | | |
| Lewis Combined | ↑ | ↑ | ↓ | ↓ | ↓ |
| Secretor | | | ↓ | | ↓ |
| Non-secretor types | ↑ | | ↓ | ↓ | ↓ |

Fucose and mannose levels were not significantly different in preterm and term human milk, either shortly after birth or at term adjusted age.

Preterm human milk (d. 1-6) from Lewis non-secretor type vs. secretor mothers had significantly less fucose. Breast milk obtained at term adjusted age had significantly less fucose from non-secretor type mothers for all infants in the study (p=0.002, data not shown), and specifically preterm infants.

Preterm human milk monosaccharide content is significantly different from breast milk at term or term-adjusted age. Maternal Lewis phenotype is significantly associated with preterm human milk monosaccharide content, indicating a genetic/dietary link.

Inventor's related study shows that maternal Lewis blood-group recessive phenotype is associated with preterm delivery (Szabo, 2008); while these data show that preterm milk monosaccharide content is significantly different from breast milk at term or term adjusted age. This could account for some of the differences in immune function and brain development observed between preterm and term infants.

### Example 4

### Monosaccharide Composition of Human Milk and Infant Formulas

### INTRODUCTION

One major difference between mother's milk and infant formula is human milk oligosaccharides (HMOs), which contain monosaccharides important in cell interaction and communication. HMOs are metabolized in the infant to provide important monosaccharide substrates; however, this may be limited in premature infants because of immature hepatic and renal function. Inventor's is the first study to evaluate the monosaccharide composition of breast milk from preterm (P) and term (T) infants of different gestational and postnatal ages, and compare it to preterm and term infant formulas.

Methods

Inventor studied 35 breast-feeding mothers to determine monosaccharide content of expressed breast milk for 23 preterm (30-37 wks) and 12 term infants (≧38 wks). Mothers expressed and collected d 1-6, d 7-10, and 1 mo. samples of breast milk, which were frozen and stored until HPLC monosaccharide analysis.

Results

Inventor determined between group breast milk comparisons, and the following table shows trends as gestation progressed towards term (30 to ≧38 wks).

| | Glc | Gal | Fuc | GlcNH₂ | Man | Neu5Ac | GalNH₂ |
|---|---|---|---|---|---|---|---|
| 1-6 d | ↓ | = | ↑ | ↑ | ↑ | ↓ | ↑ |
| 7-10 d | ↑ | ↑ | ↑ | ↑ | ↑ | = | = |
| 1 month | = | = | = | ↑ | ↑ | [symbol] | ↑ |

Glucose (Glc);
Galactose (Gal);
Fucose (Fuc);
Glucosamine (GlcNH₂);
Mannose (Man);
Sialic Acid (Neu5Ac);
Galactosamine (GalNH₂)

At 1-6 d, breast milk Glc and Neu5Ac levels were higher in P infants (p<0.05) while Fuc, GlcNH₂, Man and GalNH₂ levels were lower (p<0.05). By one month, breast milk Glc, Gal, and Fuc levels were not significantly different in P and T milks while GlcNH₂, Man, and GalNH₂ levels remained lower (p<0.05) and Neu5Ac levels remained equal or higher in preterm breast milk. When Inventor analyzed milk monosaccharide levels at term-adjusted age, Inventor found that Glc and Gal were higher in preterm milk (p=0.003, 0.029, respectively) and GlcNH₂, Neu5Ac and GalNH₂ were less (p≦0.007) in preterm milk. Infant formulas (preterm and post-discharge, term, and soy) contained only Glc and Gal.

### CONCLUSIONS

This study suggests that monosaccharide supplementation of infant formulas to levels more closely resembling sugar concentrations found in breast milk might prove beneficial to infant growth, CNS and immune function development in preterm and term infants.

### Example 5

### Monosaccharide Composition of Human Breast Milk and Infant Formulas in Preterm and Term Infants

Purpose of Study

Relative to commercial infant formulas, mother's milk has been shown to offer unique advantages to term and preterm infants with regard to immunologic processes (Hanson, 1998; Hylander, 1998) and cognitive development (Morley et al., 1988; Lucas et al., 1992, 1994, 1996). One major difference between mother's milk and infant formula is human milk oligosaccharides (HMOs), which contain monosaccharides important in cell interaction and communication. With the exception of glucose, there is little information regarding the role of dietary sugars in mothers and infants or their availability in expressed breast milk. Fucose, glucosamine, mannose, and sialic acid have all been shown to be essential to various biological activities, including immunologic and neurologic function. HMOs are metabolized in the infant to provide these additional monosaccharide substrates; however, this may be limited in premature infants because of immature hepatic and renal function. Inventor's is the first study to evaluate the monosaccharide composition of breast milk from preterm and term infants of different gestational and postnatal ages, and compare it to preterm and term infant formulas.

Methods

Subjects and Design

Inventor enrolled 35 breast-feeding mother-infant pairs: 23 preterm (30-31, 32-33, 34-35, 36-37 wks gestation) and 12 term (38-39, 40-41 wks gestation). Mothers were excluded for significant alcohol, tobacco or drug use; significant medical complications (infection, diabetes, chronic hypertension, pre-eclampsia); or chronic medication use (anti-depressants, etc.). Infants were excluded for major illness, congenital or surgical anomalies.

Mothers expressed and collected their initial breast milk samples at day 1-6 following delivery, immediately before feeding the infant. Additional samples were collected at day 7-10 and 1 month following delivery. Individual samples were frozen as soon after collection as possible, and stored until monosaccharide analyses were performed using standardized techniques for anion exchange chromatography (Mannatech, Coppell, Tex.). Infant formula samples included preterm and preterm post-discharge, term and soy. Individual samples of hydrolyzed preterm and term breast milk and infant formulas were evaluated for levels of the following monosaccharides: glucose, galactose, fucose, glucosamine, mannose, sialic acid, and galactosamine.

Results

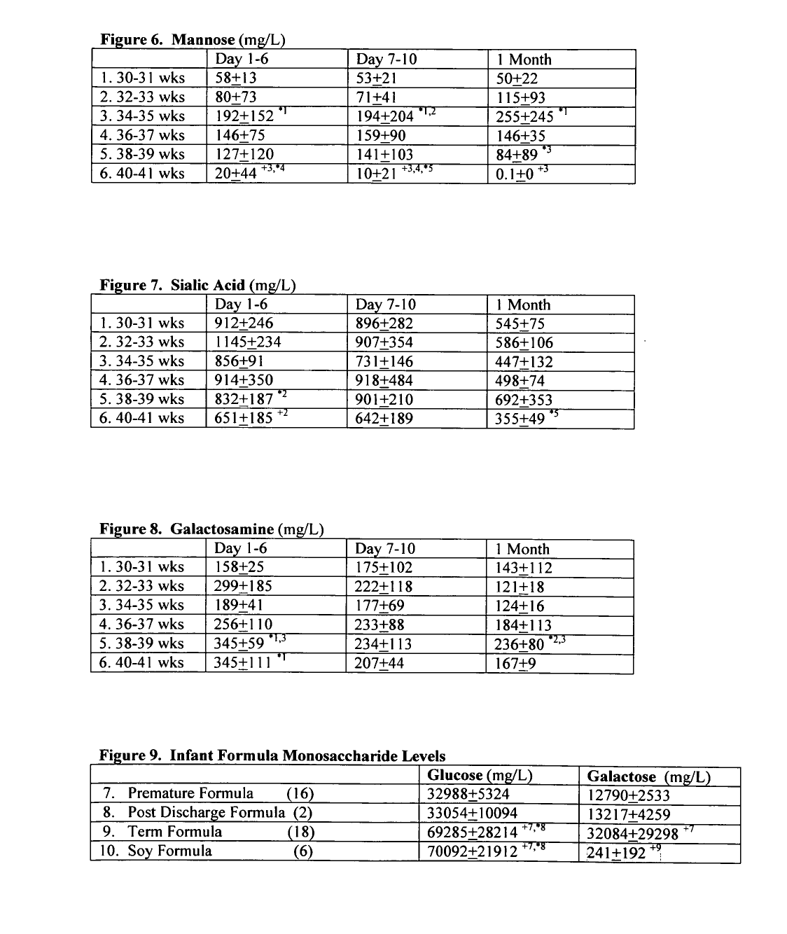

FIGS. 2-9 show breast milk values on day 1-6, day 7-10 and 1 month for glucose, galactose, fucose, glucosamine, mannose, sialic acid, and galactosamine. In FIG. 2 and subsequent figures/tables, all values are expressed as M±SD and n=( ) For between group measures, *p<0.05, +p<0.01.

Table 17 summarizes trends of each breast milk monosaccharide as gestation progresses from preterm (30-37 wks) towards term gestation (38-41 wks). At day I-6, breast milk glucose and sialic acid levels were higher in preterm infants (p<0.05) while fucose, glucosamine, mannose and galactosamine levels were lower (p<0.05). At one month, breast milk glucose, galactose, and fucose levels were not significantly different in preterm and term milk while glucosamine, mannose, and galactosamine levels remained lower (p<0.05) and sialic acid levels remained equal or higher in preterm breast milk.

TABLE 17
Breast Milk Monosaccharides

| | Glucose | Galactose | Fucose | Glucosamine | Mannose | Sialic Acid | Galactosamine |
|---|---|---|---|---|---|---|---|
| 1-6 d | ↓ | = | ↑ | ↑ | ↑ | ↓ | ↑ |
| 7-10 d | ↑ | ↑ | ↑ | ↑ | ↑ | = | = |
| 1 month | = | = | = | ↑ | ↑ | [symbol] | ↑ |

In Table 18, Inventor compared preterm breast milk samples at term adjusted age to breast milk from term infants at day 1-6 following delivery.

TABLE 18
Breast Milk Monosaccharides at Term Adjusted Age

| mg/L | Preterm (30-37 wks) | Term (38-41 wks) | p = |
|---|---|---|---|
| Glucose | 28718 ± 5650 | 21404 ± 4751 | 0.003 |
| Galactose | 27044 ± 4424 | 23381 ± 2927 | 0.029 |
| Fucose | 1777 ± 1008 | 2080 ± 1501 | 0.581 |
| Glucosamine | 4318 ± 2204 | 8192 ± 2024 | 0.0003 |
| Mannose | 131 ± 141 | 78 ± 105 | 0.316 |
| Sialic Acid | 514 ± 176 | 749 ± 200 | 0.007 |
| Galactosamine | 152 ± 65 | 345 ± 82 | 0.000 |

Inventor's analyses showed that glucose and galactose levels were higher in preterm milk at term adjusted age; fucose and mannose levels were not different; and glucosamine, sialic acid and galactosamine levels were lower.

Only glucose and galactose were found in all infant formulas tested. Glucose levels in premature and premature post discharge formulas were significantly less than values in term and soy formulas. Galactose values in premature formula were significantly less than in term formula, and values in term formula were significantly higher than in premature and soy formulas.

### CONCLUSIONS

Human milk oligosaccharides are comprised of several monosaccharides that may contribute to the many beneficial effects of breast milk. Breast milk monosaccharides vary between preterm and term milk, and remain different in preterm milk at term adjusted age. With the exception of glucose and galactose, these monosaccharides are absent in infant formulas. This study suggests that monosaccharide supplementation of infant formulas to levels more closely resembling sugar concentrations found in breast milk might prove beneficial to infant growth, CNS and immune function development in preterm and term infants.

At day 1-6, breast milk glucose and sialic acid levels were higher in preterm milk (p<0.05) while fucose, glucosamine, mannose and galactosamine levels were lower (p<0.05).

At 7-10 days of life, glucose, galactose, fucose, and glucosamine levels were lower in preterm breast milk (p<0.05); and sialic acid and galactosamine levels were not significantly different between preterm and term breast milk.

At one month, breast milk glucose, galactose, and fucose levels were not significantly different in preterm and term milk while glucosamine, mannose, and galactosamine levels remained lower (p<0.05) and sialic acid levels remained equal or higher in preterm breast milk.

Human breast milk oligosaccharides provide an array of monosaccharide substrates that vary between preterm and term milk during early lactation (d 1-6 through 1 month). Of particular interest are dynamic variations in fucose, glucosamine, mannose and sialic acid—monosaccharides known to play a role in CNS and immune function development. At term adjusted age, preterm breast milk continues to have higher levels of glucose and galactose (substrates also found in infant formula), with significantly lower levels of glucosamine and sialic acid (substrates lacking in infant formulas that may be important in infant neurologic development).

Human milk oligosaccharides are composed of several monosaccharides that may contribute to the many beneficial effects of breast milk. With the exception of glucose and galactose, these monosaccharides are absent in infant formulas. This study suggests that monosaccharide supplementation of infant formulas to levels more closely resembling sugar concentrations found in breast milk would be beneficial to infant growth, CNS and immune function development in preterm and term infants.

### Example 6

A custom blend of these monosaccharides to supplement term and preterm formulas has been developed to simulate their composition in mother's milk. Based upon preliminary data, an example of the possible compositions of the term and preterm formula monosaccharide supplements added to term and preterm infant formula is shown in Table 19.

TABLE 19
Example of glyconutritional supplementation of formulas to simulate
term breast milk

| | Term Breast Milk | Term Formula | Term MS Suppl . . . | Preterm Formula | Preterm MS Suppl. |
|---|---|---|---|---|---|
| Sugar | mg/oz | mg/oz | mg/oz. | mg/oz | mg/oz |
| Glucose | 1097.6 | 1025.4 | 0 | 1420.0 | 0 |
| Galactose | 1099.3 | 1034.6 | 0 | 432.4 | 0 |
| Fucose | 89.4 | ND | 89.4 | ND | 89.4 |
| Glucosamine | 33.8 | ND | 33.8 | ND | 33.8 |
| Mannose | 27.4 | 22.6 | 4.8 | 17.4 | 10 |
| Sialic Acid | 25.4 | 7.5 | 17.9 | to be determined | to be determined |

ND = not detected

Research Design:

A separate group of infants (5 LBW premature, 5 term) is used to pilot formula supplementation with this combination of monosaccharides during their initial hospitalization and through the first 2 weeks of life. The monosaccharide supplement is added to the appropriate preterm or term formula when infants are tolerating 100 ml/kg/day of oral formula.

If babies fall within normal limits for growth, formula tolerance, and stool output according to well accepted medical standards, Inventor consides this acceptable outcome to proceed with enrollment of monosaccharide-supplemented formula fed infants.

### Specific Aim 2

### Example 7

### Longitudinal Evaluation (0-3 Yrs) of Growth, Body Composition, General Health and Morbidity in Term Control and LBW Premature Infants

A longitudinal evaluation is performed (0-3 yrs) of growth, body composition, general health and morbidity in term control and LBW premature infants assigned to each of the following feeding groups: a) mother's milk [MM], b) appropriate non-supplemented term or premature infant formula [F], or c) infant formula supplemented with additional critical monosaccharides that are contained in mother's milk [MS+F].

Research Design:

a.) Demographic, Diet, Anthropometry and Body Composition Assessments

The following data, listed in Table 20, are collected on study infants assigned to the three feeding groups:

TABLE 20
Demographic, Diet, Anthropometry and Body Composition
Assessments

| | 37-42 | 6 | 12 | 24 | 36 |
|---|---|---|---|---|---|
| | weeks | months | months | months | months |
| Demographics/updates* | x | x | x | x | x |
| Dietary Assessment | x | x | x | x | x |
| Anthropometric Data | x | x | x | x | x |
| Body Composition | x | x | x | x | x |
| BIA, DEXA | | | | | |
| Blood Analyses | x | x | x | — | — |

*see Appendix VII for details

Dietary Assessment. Human milk and formula intakes is extracted from medical record flow sheets (volumes and energy density) during initial hospitalization and from three-day dietary records (number of times fed, volume if available, energy density) collected prior to the term, 6, 12, 24, and 36 months.
Once the child is taking food (6-36 months visits), the parent is instructed on the use of the Food Record Guide, a booklet that assists the parent in the completion of the food record. The parent is given the food record to complete at home and a postage-paid envelope with instructions to return the record to the study coordinator. Nutrient analysis utilizes the Minnesota Nutrition Data System for Research (Food Nutrient and Data Base Version 4.04_{—}32).
Anthropometry and Body Composition. Anthropometric measures, e.g., weight, length or height, and head circumference are obtained using standardized techniques at 37-42 weeks, 6, 12, 24, and 36 months. Growth measures are obtained at each visit and growth velocity is calculated. Growth parameters in LBW infants are generally corrected for various periods following birth, e.g., head circumference until 18 months post-term, weight until 24 months post-term, and length until 3.5 years post-term. LBW infants have all growth parameters age corrected for prematurity through the 36 month evaluation to provide more consistency.
Body composition is determined using standardized techniques for measuring mid-arm circumference, triceps skin-fold, and calculate body mass index as appropriate. Infants and children also have BIA and DEXA studies performed.
Blood Analyses. All infants enrolled in the study have blood drawn in the hospital to determine baseline laboratory values (electrolytes, liver function, chemistries, and complete blood count as listed in Appendix V) and monosaccharide levels.

Statistical Analyses:

Separate MANOVAs and ANOVAs are computed for each variable described above.

b.) Assessment of General Health and Morbidity

To assess general health and morbidity in term and LBW premature infants assigned to each of the three feeding groups, typical in hospital and postdischarge clinical problems/morbidity data are collected and groups are compared using maximum likelihood methods for estimating the exact p-value (e.g., likelihood ratio test). Results are confirmed using the Fisher-Freeman-Halton test.

### Specific Aim 3

### Example 8

### Correlation of Cognitive/Behavioral Brain Function with Physiological Brain Function

Cognitive/behavioral brain function is correlated with physiologic brain function (0-3 yrs) in LBW premature and term infants assigned to each of the three feeding groups.

Rationale: The neuropathology (Fuller et al., 1983) and subsequent deficits associated with premature infants have been examined by several investigators (Ross et al., 1996; Dehaene-Lambertz, 1997; Fuller et al., 1983). The neuropathology in preterm infants often results in deficits affecting “higher order” cognitive and executive functions, language, visual attention, visual-motor control, memory and memory consolidation, fine and gross motor control, and internal inhibitory control. The complex cognitive and attention deficits which can occur in as many as 25% to 50% of small premature infants may have no accompanying motor deficits, and may not be easily attributed entirely to white matter disease. Higher cortical function deficits may be disturbed secondary to cerebral cortical disturbances, such as aberrations of cortical connectivity and synapse formation (Ross et al., 1996; Volpe, 1996).

Language development is one of the most reliable predictors of intelligence and can be used to help identify children with cognitive impairment (Capute et al., 1986). Socioeconomic status has been reported to predict language ability and recovery of attention to a novel stimulus independent of other predictors, but systematic, prospective studies in this area remain to be done. Medical complications besides intraventricular hemorrhage may also make an important independent contribution to the variance in language outcome (Lewis and Bendersky, 1989).

Developmental and neuropsychologic evaluations are done in order to assess the role of nutrition in predicting cognitive and behavioral outcome in term and LBW premature infants.

A.) Cognitive/Behavioral Brain Function

Research Design:

The following data on study infants assigned to the three feeding groups are collected. The studies, listed in Table 21, are among the Cognitive/Behavioral Assessments to be performed in the brain function laboratory:

TABLE 21
Cognitive/Behavioral Assessments*

| | 6 months | 12 months | 24 months | 36 months |
|---|---|---|---|---|
| Preschool Language | x | x | x | x |
| Scale-3 | | | | |
| Bayley Scales of Infant | x | x | x | — |
| Development | | | | |
| Battelle Developmental | x | x | x | x |
| Inventory | | | | |
| Carey Temperament | x | x | x | x |
| Scales | | | | |
| Child Behavior Checklist | — | — | x | x |
| Purdue Pegboard Test | — | — | — | x |
| Stanford Binet IV | — | — | — | x |
| NEPSY | — | — | — | x |

Statistical Analyses:

The analyses here involve repeated measures MANOVAs and ANOVAs as described hereinabove.

B.) Physiologic Brain Function

Research Design:

The following data are collected on study infants assigned to the three feeding groups. The studies, listed in Table 22, are among the psychophysiologic assessments to be performed in the brain function laboratory:

TABLE 22
Psychophysiologic Assessments*

| | 37-42 weeks | 6 months | 12 months | 24 months | 36 months |
|---|---|---|---|---|---|
| Grasp Force Measurement | x | x | x | x | x |
| Finger-Length Pattern Measurement | x | x | x | x | x |
| Baseline/Social Test | — | x | x | — | — |
| Baseline/Startle Test | x | x | x | x | x |
| Auditory Lang. Discrimination | x | x | x | x | x |
| Auditory Lang. Discrimination Ext. | — | — | — | — | x |
| Visual Attention Test | — | — | — | — | x |
| Continuous Performance Test | — | — | — | — | x |
| Post-Feeding Rest Recordings | x | x | x | — | — |

Psychophysiologic assessments are done initially at term corrected age and subsequently at 6, 12, 24, and 36 months corrected age for LBW premature infants and chronologic age for term infants.

Heart rate, skin conductance, respiration, chin muscle activity (EMG), eye movements (EOG) and brain waves (EEG) during testing conditions are continually recorded.

Statistical Analyses:

The analyses planned here are more complicated than those described above because of the multiplicity of responses (e.g., variations in behavioral state, EEG frequencies and ERP waves). Groups can be compared as described above with the standard repeated measures MANOVAs and ANOVAs after the data are grouped into relevant categories. Every effort is made to reduce the multiplicity of statistical tests by constructing composite variables using PCA/Promax analyses.

### Specific Aim 4

### Example 9

### Assessing the Influence of Family and Home Environment

The influence of family and home environment on developmental outcome of LBW premature and term infants assigned to each of the three feeding groups to determine the role of environment vs. nutrition on each of the study variables is assessed.

Research Design:

The following data on study infants assigned to the three feeding groups are collected. The studies, listed in Table 23, are among the standardized Home and Family Assessments performed in the brain function laboratory.

Statistical Analyses:

The personality measures are included in the study to identify parents who have a serious pathology, making it possible to look at their children in relation to the others when the study is over, using nonparametric statistics. Since all the data to be obtained under this aim are at least ordinal, it is possible to do the MANOVAs and ANOVAs on each as described above for other measures. The correlations of all measures in Table 23 are computed and factor analyzed if there are many correlations 0.50 or above.

TABLE 23
Home and Family Assessments*

| | 6 | 12 | 24 | 36 |
|---|---|---|---|---|
| | months | months | months | months |
| Wechsler Abbreviated Scale | — | x | — | — |
| Of Intelligence (WASI) | | | | |
| Family Adaptability and Cohesion | x | x | x | x |
| Evaluation Scales-II (FACES-II) | | | | |
| Family Inventory of Life Events | x | x | x | x |
| And Changes (FILE) | | | | |
| Hollingshead Four Factor Index | x | x | x | x |
| Of Social Position | | | | |
| Symptom Assessment-45 | x | x | x | x |
| Questionnaire (SA-45) | | | | |
| Minnesota Multiphasic Personality | x | — | — | — |
| Inventory-2 (MMPI-2) | | | | |
| PROCESS | x | x | x | x |
| Edinburgh Handedness Inventory | x | — | — | — |

### Specific Aim 5

### Example 10

### Comparison of Term Siblings of the LBW Premature vs. Term Babies

The objective is to compare term siblings of the LBW premature vs. term babies at age 3 yrs. to provide preliminary complementary information about the calculated estimate of heritability influencing the nutrition-development relationship.

Research Design:

Siblings of the same parentage (˜20 each for LBW and control groups) that will reach 3 years of age during the study period are assessed once, at about the age of three years (±1 month). Siblings enrolled in this study are healthy, with weight within normal range for age.

Statistical Analyses:

Physiological data gathered in this study are written to digital disks for archiving. Performance data from paradigms (e.g., reaction times and accuracy of responses), nutritional and psychological survey data, and general demographic data, are entered into a database with an Access front end and an SQL back end. Analysis is accomplished with tools like Excel, SAS, SPSS, BMDP, and SYSTAT.

Grasp force data (averages/hand of isometric force values) is used to compare left-right hand strength differences.

Finger-length data (ratios of second to fourth finger lengths) is related to other lateralization measures.

Resting state recordings are evaluated using standardized scoring criteria for state determination (Anders et al., 1971) based on EEG, EMG and EOG data coupled with quantitative analyses of EEG, autonomic activity and video recordings.

Heart rate analyses during resting periods and in association with stimuli is measured in terms of beat-to-beat intervals (R-R).

Respiratory rate is analyzed in the time domain and is measured by the fraction of a cycle occurring in comparable periods of time before and after a stimulus. Groups are contrasted in terms of their mean responses in various periods of time.

Skin conductance is measured as the conductance change from stimulus onset to peak.

General Statistical Considerations. The distributions of all variables are inspected separately and all variables with skewed distributions are transformed (log, square root, etc.). Statistical analyses of quantitative EEG procedures, heart period, respiratory rate, and skin conductance include correlational analyses, factor analyses, MANOVAs and ANOVAs as described above.

### DISCUSSION

The abovementioned examples define new methods and compositions to aid proper development of growth, cognitive and immunologic systems in LBW preterm versus term infants. Furthermore, relative dosage amounts and delivery times are indicated.

While the present invention has now been described in terms of certain preferred embodiments, and exemplified with respect thereto, one skilled in the art will readily appreciate that various modifications, changes, omissions and substitutions may be made without departing from the spirit thereof. It is intended, therefore, that the present invention be limited solely by the scope of the following claims.

### REFERENCES

Alton G, Hasilik M, Niehues R, Panneerselvan K, Etchison J R, Fana F, Freeze H H. Direct utilization of mannose for mammalian glycoprotein biosynthesis. Glycobiology. 1998; 8:285-295
Alton G, Kjaergaard S, Etchison J R, Skovby F, Freeze H H. Oral ingestion of mannose elevates blood mannose levels: a first step toward a potential therapy for carbohydrate-deficient glycoprotein syndrome type I. Biochem Mol. Med. 1997; 60:127-133
Aminoff D. Methods for the quantitative estimation of N-acetylneuraminic acid and their application to hydrolysates of sialomucoids. Biochem J. 1961:81:384-392
Anderson J W. Nutritional management of diabetes mellitus. In: Modern Nutrition in Health and Disease (Shills, M. E., Olsen, J. A., Shike, M. & Ross A. C., eds), 1998; pp. 1365-1394, Lippincott.
Babbar H S, Jaswal V M, Gupta R, Mahmood A. Intestinal absorption of macromolecules and epithelial cell surface glycosylation in suckling rats nursed on mothers fed low-protein diet-I. Biol Neonate. 1990; 58:104-111
Battaglia F C. Placental transport and utilization of amino acids and carbohydrates. Fed Proc. 1986; 45:2508-2512.
Baumbach G A, Saunders P T, Ketcham C M, Bazer R W, Roberts R M. Uteroferrin contains complex and high mannose-type oligosaccharides when synthesized in vitro. Mol Cell Biochem. 1991; 105:107-117.
Bell D J, Talukder M Q. Rates of urinary excretion of free aldopentoses and fucose by fasting healthy adults: effects of ingested pentose-containing foods. Clin Chim Acta. 1972; 4013-4020
Berger V, Perier S, Pachiaudi C, Normand S, Louisot P, Martin A. Dietary specific sugars for serum protein enzymatic glycosylation in man. Metabolism. 1998; 47:1499-1503
Beuth J, Ko Hl, Pulverer G, Uhlenbruck G, Pichlmaier H. Importance of lectins for the prevention of bacterial infections and cancer metastases. Int J Med Microbiol Virol Parasitol Infect Dis. 1994; 281:324-333
Bihler I. Intestinal sugar transport: ionic activation and chemical specificity. Biochim Biophys Acta. 1969; 183:169-181
Bocci V, Winzler R J. Metabolism of L-fucose-1-¹⁴C and of fucose glycoproteins in the rat. Am J. Physiol. 1969; 216:1337-1342
Brand-Miller J, Bull S, Miller J, McVeagh P. The oligosaccharide composition of human milk: temporal and individual variations in monosaccharide components. J Pediatr Gastroenterology and Nutr. 1994; 19:371-376
Brown E, Hughes R C, Watts R W. Biochemical expression of the galactosemic defect in lymphocytes and the effects on glycoprotein synthesis. Metabolism. 1977; 26:1047-1055
Brunngraber E G, Brown B D, Aro A. Distribution and age-dependent concentration in brain tissue of glycoproteins containing N-acetylgalactosamine. Neurobiol. 1975; 5:339-346
Brunngraber E G, Davis L G, Javaid J I, Berra B. Glycoprotein catabolism in brain tissue in the lysosomal enzyme deficiency diseases. Adv Exp Med. Biol. 1976; 68:31-48
Brydon W G, Merrick M V, Hannan J. Absorbed dose from ¹⁴C xylose and ¹⁴C mannose. Br J. Radiol. 1987; 60:563-566
Buchmiller T L, Fonkalsrud E W, Kim C S, Chopourian H L, Shaw K S, Lam M M, Diamond J M. Upregulation of nutrient transport in fetal rabbit intestine by transamniotic substrate administration. J Surg Res. 1992; 52:443-447.
Capps J C, Shetlar M R, Bradford R H. Hexosamine metabolism. I. The absorption and metabolism, in vivo, of orally administered D-glucosamine and N-acetyl-D-glucosamine in the rat. Biochim Biophys Acta. 1966; 127:194-204
Carlson S E, House S G. Oral and intraperitoneal administration of N-acetylneuraminic acid: effect on rat cerebral and cerebellar N-acetylneuraminic acid. J. Nutr. 1986; 116:881-886
Carlson S E. N-Acetylneuraminic acid concentrations in human milk oligosaccharides and glycoproteins during lactation. Am J Clin Nutr. 1985; 41:720-726
Carstensen M, Leichweiss H P, Molsen G, Schroder H. Evidence for a specific transport of D-hexoses across the human term placenta in vitro. Arch Gynakol. 1977; 222:187-196.
Charlton V E, Reis B L. Effects of gastric nutritional supplementation on fetal umbilical uptake of nutrients. Am J. Physiol. 1981; 241:E178-185.
Cooper R, Osselton J, Shaw J. EEG Technology, Ed. 3^{rd}, 1980, Butterworths, London.
Cox F, Taylor L. Prevention of Escherichia coli K1 bacteremia in newborn mice by using topical vaginal carbohydrates. J Infect Dis. 1990; 162:978-981
Dickson J, Messer M. Intestinal neuraminidase activity of suckling rats and other mammals. Biochem J. 1978; 170:407-413
Druse M J, Tonetti D A, Smith D M Jr. Effects of maternal nutritional stress on synaptic plasma membrane proteins and glycoproteins in offspring. Exp Neurol. 1982; 78:99-111
Druse M J, Tonetti D A, Smith D M Jr. Effects of maternal nutritional stress on synaptic plasma membrane proteins and glycoproteins in offspring. Exp Neurol. 1982; 78:99-111
Dwek R A, Lellouch A C, Wormald M R. Glycobiology: the function of sugar in the IgG molecule. J. Anat. 1995; 187:279-292
Eberendu A R, Booth C, Luta G, Edwards J A, McAnalley B H. Quantitative determination of saccharides in dietary glyconutritional products by anion-exchange liquid chromatography with integrated pulsed amperometric detection, J AOAC International. 2005; 88:998-1007.
Egge H, Dell A, Von Nicolai H. Fucose-containing oligosaccharides from human milk. Arch Biochem Biophys. 1983; 224:235-253
Ehrhardt R A, Bell A W. Developmental increases in glucose transporter concentration in the sheep placenta. Am J. Physiol. 1997; 273:R1132-R1141.
Etchison J R, Freeze H H. Enzymatic assay of D-mannose in serum. Clinical Chemistry. 1997; 43:533-538
Floch F, Werner G H. In vivo antiviral activity of D-glucosamine. Arch Virol. 1976; 52:169-173
Flowers H M. Chemistry and biochemistry of D- and L-fucose. Adv Carbohydr Chem Biochem. 1981; 39:279-345
Fowden A L, Forhead A J, Silver M, MacDonald A A. Glucose, lactate and oxygen metabolism in the fetal pig during late gestation. Exp Physiol 1997; 82:171-182.
Freeze H H. Disorders in protein glycosylation and potential therapy: tip of an iceberg? J. Pediatr. 1998; 133:593-600
Gannon M C, Khan M. A, Nuttall F Q. Glucose appearance rate after the ingestion of galactose. Metabolism. 2001; 50: 93-98.
Gardiner, T H. 2004 Pharmacokinetics and safety of glyconutritional sugars for use as dietary supplements. GlycoScience and Nutrition 5(2):1-11
Ginsberg G, Slikker W Jr, Bruckner J, Sonawane B. Incorporating children's toxicokinetics into a risk framework. Environ Health Perspect. 2004 February; 112(2):272-83. Review.
Greenhouse, S. W. & Geisser, S. On methods in the analysis of profile data. Psychometrica. 1959; 24:95-112
Hading V J, Nicholson T F, Armstrong A R. Cutaneous blood-sugar curves after the administration of fructose, mannose and xylose. Biochem J. 1933; 128:2035-2042 Hanson L A. Breastfeeding provides passive and likely long-lasting active immunity. Ann Allergy Asthma Immunol. 1998; 81:523-533
Hanson L A. Breastfeeding provides passive and likely long-lasting active immunity. Ann Allergy Asthma Immunol. 1998; 81:523-533
Hay W W Jr. Regulation of placental metabolism by glucose supply. Reprod Fertil Dev. 1995; 7:365-375.
Hay W W Jr., Sparks J W, Wilkening R B, Battaglia F C, Meschia G. Fetal glucose uptake and utilization as functions of maternal glucose concentration. Am J Physiol 1984; 246:E237-E242.
Hayakawa K, De Felice C, Watanabe T, Tanaka T, Iinuma K, Nihei K, Higuchi S, Ezoe T, Hibi I, Kurosawa K. Determination of free N-acetylneuraminic acid in human body fluids by high-performance liquid chromatography with fluorimetric detection. J. Chromatogr. 1993; 620:25-31
Hylander M A, Strobino D M, Dhanireddy R. Human milk feedings and infection among very low birth weight infants. Pediatrics. 1998; 102:E38
Icagasioglue D, Caksen H, Yildiz B, Cetinkaya O, Sialic acid levels in healthy preterm and fullterm infants. Indian Pediatr. 2001; 38:1429
Kamel M, Hanafi M, Bassiouni M. Inhibition of elastase enzyme release from human polymorphonuclear leukocytes by N-acetyl-galactosamine and N-acetyl-glucosamine. Clin Exp Rheumatol 1991; 9:17-21
Kaufman R L, Ginsburg V. The metabolism of L-fucose by HeLa cells. Exp Cell Res. 1968; 50:127-132
Kingham J G, Baker J H, Loehry C A. Autoradiographic study of the permeability characteristics of the small intestine. Gut. 1978; 19:114-120
Koch A, Melbye M, Sorensen P, Homoe P, Madsen H O, Molbak K, Hansen C H, Andersen L H, Hahn G W, Garred P. Acute respiratory tract infections and mannose-binding lectin insufficiency during early childhood. JAMA. 2001; 285:1316-1321
Kunz C, Rudloff S. Biological functions of oligosaccharides in human milk. Acta Paediatr. 1993; 82:903-912
Kuranda M J, Aronson N N, Jr. Use of active site-directed inhibitors to study in situ degradation of glycoproteins by the perfused rat liver. J Biol. Chem. 1985; 260:1858-1866
Lau Y L, Chan S Y, Turner M W, Fong J, Karlberg J. Mannose-binding protein in preterm infants: developmental profile and clinical significance. Clin Exp Immunol. 1995; 102:649-654
Lemonnier M, Bourrillon R. Low-molecular-weight carbohydrate-rich compounds in pregnancy urine. Biomedicine. 1976; 24:253-258
Lemonnier M, Derappe C, Bourrillon R. Sialic acid-containing glycoconjugates in human lactation urine. Biomedicine. 1978; 29:146-150
Lipscombe R J, Lau Y L, Levinsky R J, Sumiya M, Summerfield J A, Turner M W. Identical point mutation leading to low levels of mannose binding protein and poor C3b mediated opsonisation in Chinese and Caucasian populations. Immunology Letters; 1992; 32:253-258
Lucas A, Fewtrell M S, Morley R, Lucas P J, Baker B A, Lister G, Bishop N J. Randomized outcome trial of human milk fortification and developmental outcome in preterm infants. Am J Clin Nutr. 1996; 64:142-151
Lucas A, Morley R, Cole T J, Gore S M. A randomised multicentre study of human milk versus formula and later development in preterm infants. Arch Dis Child Fetal Neonatal Ed. 1994; 70:F141-146
Lucas A, Morley R, Cole T J, Lister G, Leeson-Payne C. Breast milk and subsequent intelligence quotient in children born preterm. Lancet. 1992; 339:261-264
Margolis R K, Preti C, Lai D, Margolis R U. Developmental changes in brain glycoproteins. Brain Res. 1976; 112:363-369
Marquardt T, Luhn K, Srikrishna G, Freeze H H, Harms E, Vestweber D. Correction of leukocyte adhesion deficiency type II with oral fucose. Blood. 1999; 94:3976-3985
Martin A, Rambal C, Berger V, Perier S, Louisot P. Availability of specific sugars for glycoconjugate biosynthesis: a need for further investigations in man. Biochimie. 1998; 80:75-86
McVeagh P, Brand Miller J. Human milk oligosaccharides: only the breast. J Paediatr Child Health. 1997; 33:281-286
Meschia G, Battaglia F C, Hay W W, Sparks J W. Utilization of substrates by the ovine placenta in vivo. Fed Proc. 1980; 39:245-249.
Michaels E K, Chmiel J S, Plotkin B J, Schaeffer A J. Effect of D-mannose and D-glucose on Escherichia coli bacteriuria in rats. Urol Res. 1983; 11:97-102
Morgan B, Winick M. Effects of administration of N-acetylneuraminic acid (NANA) on brain NANA content and behavior. J. Nutr. 1980; 110:416-424
Morgan B, Winick M. The subcellular localization of administered N-acetylneuraminic acid in the brains of well-fed and protein restricted rats. Br J. Nutr. 1981; 46:231-238
Morley R, Cole T J, Powell R, Lucas A. Mother's choice to provide breast milk and developmental outcome. Arch Dis Child. 1988; 63:1382-1385
Murray, R. K., Granner, D. K., Mayes, P. A. and Rodwell, V. W. (eds). Harper's Illustrated Biochemistry. Ed. 26^{th} Lange Medical Books/McGraw-Hill, New York, N.Y., 2003 pp. 474, 167, 514-534.
Newburg D S, Pickering L K, McCluer R H, Cleary T G. Fucosylated oligosaccharides of human milk protect suckling mice from heat-stabile enterotoxin of Escherichia coli. J Infect Dis. 1990; 162:1075-1080
Niehues R, Hasilik M, Alton G, Korner C, Schiebe-Sukumar M, Koch H G, Zimmer K P, Wu R, Harms E, Reiter K, von Figura K, Freeze H H, Harms H K, Marquardt T. Carbohydrate-deficient glycoprotein syndrome type Ib. Phosphomannose isomerase deficiency and mannose therapy. J Clin Invest. 1998; 101:1414-1420
Olivieri W, Wolverton C K, Ruwe P J, White M E, Ransay T G. Comparison of the effects of dietary glucose versus galactose on porcine feto-placental glucose metabolism. J. Nutr. 1990; 120:1241-1247.
Pennell P B. Antiepileptic drug pharmacokinetics during pregnancy and lactation. Neurology. 2003; 61:S35-S42
Pere M C. Effects of meal intake on materno-foetal exchanges of energetic substrates in the pig. Reprod Nutr Dev. 2001; 41:285-296
Pere M C. Matemo-foetal exchanges and utilisation of nutrients by the foetus: comparison between species. Reprod Nutr Dev. 2003; 43:1-15.
Perez M C, Vilas P, Perez P S, Martin A. Antiviral activity of a D-glucosamine derivative against herpetic ulcers (HSV type 2) in rabbit cornea. Acta Ophthalmol. 1989; 67:55-60
Petry, K. G. & Reichardt, J. K. (1998) The fundamental importance of human galactose metabolism: lessons from genetics and biochemistry. Trends in Genetics. 1998; 14:98-102.
Phillips J D, Diamond J M, Fonkalsrud E W. Fetal rabbit intestinal absorption: implications for transamniotic fetal feeding. J Pediatr Surg. 1990; 25:909-913.
Phillips J D, Fonkalsrud E W, Mirzayan A, Kim C S, Kieu A, Zeng H, Diamond J M. Uptake and distribution of continuously infused intraamniotic nutrients in fetal rabbits. J Pediatr Surg. 1991; 26:374-378.
Quraishi A N, Illsley N P. Transport of sugars across human placental membranes measured by light scattering. Placenta 1999; 20:167-174.
Revilla N B, Rodriguez Aparicio L B, Ferrero M A, Reglero A. Regulation of capsular polysialic acid biosynthesis by N-acetyl-D-mannosamine, an intermediate of sialic acid metabolism. FEBS Lett. 1998; 426:191-195
Roberts R M, Baumbach G A, Saunders P T, Raub T J, Renegar R H, Bazer F W. Possible function of carbohydrate on glycoproteins secreted by the pig uterus during pregnancy. Mol Cell Biochem. 1986; 72:67-79.
Sabharwal H, Sjoblad S, Lundblad A. Sialylated oligosaccharides in human milk and feces of preterm, full-term, and weaning infants. J Pediatr Gastroenterol Nutr. 1991; 12:480-484

Sanchez-Diaz A, Ruano M J, Lorente F, Hueso P. A critical analysis of total sialic acid and sialoglycoconjugate contents of bovine milk-based infant formulas. J Pediatr Gastroenterol Nutr. 1997; 24:405-410
Sasaki H, Momoi T, Yamanaka C, Kaji M, Mikawa H. Developmental changes of plasma ganglioside concentration during the neonatal period. Early Hum Dev. 1989; 20:143-150
Saunders P T, Renegar R H, Raub T J, Baumbach G A, Atkinson P H, Bazer F W, Roberts R M. The carbohydrate structure of porcine uteroferrin and the role of the high mannose chains in promoting uptake by the reticuloendothelial cells of the fetal liver. J Biol. Chem. 1985; 260:3658-3665.
Schauer R, Kamerling J P. Chemistry, biochemistry and biology of sialic acids. In: Montreuil J, Vliegenthart J F G, Schachter H, eds. Glycoproteins II. Amsterdam: Elserier Science BV; 1997 pp 244-269
Schauer R. Chemistry, metabolism, and biological functions of sialic acids. Adv Carbohydr Chem. Biochem. 1982; 40:131-234
Schroder H, Leichtweiss H P, Madee W. The transport of D-glucose, L-glucose and D-mannose across the isolated guinea pig placenta. Pflugers Arch. 1975; 356; 267-175.
Sharma P K, Garg S K, Narang A. Pharmacokinetics of oral ibuprofen in premature infants. J Clin Pharmacol. 2003 September; 43(9):968-973
Silverman M, Aganon M A, Chinard F P. Specificity of monosaccharide transport in dog kidney. Am J Physiol 1970; 218:743-750
Skoza L, Mohos S. Stable thiobarbituric acid chromonthphore with dimethyl sulphoxide. Application to sialic acid assay in analytical de-O-acetylation. Biochem J. 1976; 159:457-462
Stahl P D. The macrophage mannose receptor: current status. Am J Respir Cell Mol Biol. 1990; 2:317-318
Stahl P D. The mannose receptor and other macrophage lectins. Curr Opin Immunol. 1992; 4:48-52
Staud F, Fendrich Z, Karlicek R, Pavek P. Unidirectional transfer of D-xylose across the rat placenta. Clin Exp Pharmacol Physiol. 1998; 25:54-56.
Summerfield J A, Sumiya M, Levin M, Turner M W. Association of mutations in mannose binding protein gene with childhood infection in consecutive hospital series. BJL. 1997; 314:1229-1237
Takata I, Chida K, Gordon M R, Myrvik Q N, Ricardo M J J, Kucera L S. L-fucose, D-mannose, L-galactose, and their BSA conjugates stimulate macrophage migration. L Leukoc Biol. 1987; 41:248-256
Talent J M, Gracy R W. Pilot study of oral polymeric N-acetyl-D-glucosamine as a potential treatment for patients with osteoarthritis. Clin Ther. 1996; 18:1184-1190
Tamburrano G, Lala A., Locuratol N, Leonetti F, Sbraccia P, Giaccari A, Busco S, Porcu S. Electroencephalography and visually evoked potentials during moderate hypoglycemia. Journal of Clinical Endocrinology and Metabolism. 1988; 66:1301-1306
Teng C C, Tjoa S, Fennessey P V, Wilkening R B, Battaglia F C. Transplacental carbohydrate and sugar alcohol concentrations and their uptakes in ovine pregnancy. Exp Biol Med. 2002; 227:189-195.
Terai I, Kobayashi K. Perinatal changes in serum mannose-binding protein (MBP) levels. Immunol Lett. 1993; 38:185-187
Tram T, Brand Miller J C, McNeil Y, McVeagh P. The sialic acid content of infant saliva: comparison of human milk-fed and formula fed infants. Arch Dis Child. 1997; 77:315-318
Tsukada M, Spicer S S. Heterogeneity of macrophages evidenced by variability in their glycoconjugates. J Leukocyt Biol. 1988; 43:455-467
Viverge D, Grimmonprez L, Cassanas G, Bardet L et al. Discriminant carbohydrate components of human milk according to donor secretor types. J Pediatr Gastroenterol. 1990; 11:365-370
Viverge D, Grimmonprez L, Cassanas G, Bardet L, Bonnet H, Solere M. Variations of lactose and oligosaccharides in milk from women of blood types secretor A or H, secretor Lewis, and secretor H/nonsecretor Lewis during the course of lactation. Ann Nutr Metab. 1985; 29:1-11.
Wang B, Brand-Miller J, McVeagh P, Petocz P. Concentration and distribution of sialic acid in human milk and infant formulas. Am J Clin Nutr. 2001; 74:510-515.
Wang C, Szabo J S, Dykman R A. Effects of a carbohydrate supplement upon resting brain activity. Integrative Physiol & Behav Sci. 2004; 39:126-138
Warren L. The thiobarbituric acid assay of sialic acids. J Biol. Chem. 1959; 234:1971-1975
Webster J C, Klingman J D. Incorporation of radiolabelled sugars into synaptic junctional macromolecules from chick brain. Brain Res Bull. 1980; 5:31-34
Whyte R K, Horner R, Pennock C A. Faecal excretion of oligosaccharides and other carbohydrates in normal neonates. Arch Dis Child. 1978; 53:913-915
Wiese T J, Dunlap J A, Yorek M A. Effect of L-fucose and D-glucose concentration on L-fucoprotein metabolism in human Hep G2 cells and changes in fucosyltransferase and alpha-L-fucosidase activity in liver of diabetic rats. Biochim Biophys Acta. 1997; 1335:61-72
Wiese T J, Dunlap J A, Yorek M A. L-fucose is accumulated via a specific transport system in eukaryotic cells. J Biol. Chem. 1994; 269:22705-22711
Wood C F, Cahill G F. Mannose utilization in man. J Clin Invest. 1963; 42:1300-1312
Wullenweber M, Beutin L, Zimmermann S, Jonas C. Influence of some bacterial and host factors on colonization and invasiveness of Escherichia coli K1 in neonatal rats. Infect Immun. 1993; 61:2138-44
Yamaguchi M, Sakata M, Ogura K, Miyake A. Gestational changes of glucose transporter gene expression in the mouse placenta and decidua. J Endocrinol Invest. 1996; 19:567-569.
Yang H-J, Hakomori S-I. A sphingolipid having a novel type of ceramide and lacto-N-fucopentaose III. J Biol. Chem. 1971; 246:1192-1200
Yki J H, Virkamaki A, Daniels M C, McClain D, Gottschalk W K. Insulin and glucosamine infusions increase O-linked N-acetyl-glucosamine in skeletal muscle proteins in vivo. Metabolism. 1998; 47:449-455
Zubay G. Biochemistry. 3^{rd} ed. Dubuque, Iowa: Wm. C. Brown Publishers; 1993 p 587
Thurl et al., 1997, Glyconconj J, 14:795-799.
Nakhla et al., 1999, British J Nutr, 82:361-367.
Erney et al., 2001, Bioactive Components of Human Milk. Plenum Publishers, New York.
D'Adamo and Kelly, 2001, Alternative Med Rev, 6:390-405.
Kobata, 2004, Arch Biochem Biophys, 426:107-121.
LePendu, 2004, Adv Exp Med Biol, 554:235-243.

What is claimed is:

1. A method for determining the effect of critical nutrient dietary supplements in premature and term infants on growth, health, physiologic and cognitive/behavioral brain function during early childhood development, said method comprising:
determining a critical target nutrient content profile in typical samples of preterm and term mother's milk (MM);
developing a supplement comprising missing critical target nutrients (MS);
adding the supplement to an infant's diet, in order to simulate mother's milk;
performing a longitudinal physical evaluation of early child development;
correlating physiologic brain function with cognitive/behavioral brain function in low birth weight (LBW) premature and term infants assigned to MM, F, or MS+F during infancy, from birth through 3 years;
assessing influence of family and home environment on developmental outcome of premature and term infants assigned to MM, F, or MS+F in order to determine the role of environment vs. nutrition on growth, health, physiologic and cognitive/behavioral brain functioning; and
comparing term siblings of LBW premature vs. term babies at age 3 years in order to provide preliminary complementary information about heritability influencing the nutrition-development relationship.

2. A method for determining effectiveness of critical monosaccharide dietary supplements on growth, health, physiologic and cognitive/behavioral brain function in premature and term infants during early childhood development, said method comprising:
determining a monosaccharide content profile in typical samples of preterm and term mother's milk (MM);
developing supplements for missing critical monosaccharides (MS);
adding the supplements to preterm and term mothers' milk (MM) or formulas (F) in order to simulate their composition in mother's milk (MS+F);
performing a longitudinal physical evaluation of early child development;
correlating physiologic brain function with cognitive/behavioral brain function in premature and term infants assigned to diet groups MM, F, or MS+F during infancy;
assessing influence of family and home environment on developmental outcome of premature and term infants assigned to said MM, F, or MS+F groups in order to determine the role of environment vs. nutrition on growth, health, physiologic and cognitive/behavioral brain functioning from birth through 3 years; and
comparing term siblings of the low birth weight (LBW) premature vs. term babies at age 3 years
in order to provide preliminary complementary information about heritability influencing the nutrition-development relationship.

3. The method according to claim 2, wherein the developing comprises pharmaceutical compounding of one or more members of the group consisting of fucose, mannose, glutamic acid and sialic acid.

4. The method according to claim 2, wherein the adding is to MM and preterm and term infant F.

5. The method according to claim 2, wherein the adding is to intravenous fluids.

6. The method according to claim 2, wherein the adding is to enteral feeds.

7. The method according to claim 2, wherein the adding comprises an amount determined by bioavailability and safety in a pharmaceutically acceptable carrier.

8. The method according to claim 2, wherein the adding is supplementing in Lewis recessive phenotype mothers.

9. The method according to claim 2, wherein the adding is through the first 6 months of infancy.

10. The method according to claim 2, wherein the performing comprises a period of 0-3 years.

11. The method according to claim 2, wherein the longitudinal evaluation comprises evaluation of
growth and body composition,
general health,
immunocompetence,
incidence of infection, and
morbidity.

12. The method according to claim 11, wherein the growth and body composition is evaluated by anthropometrics.

13. The method according to claim 12, wherein the anthropometrics comprises measurements of weight, length or height, head circumference and the like using standardized techniques.

14. The method according to claim 2, wherein the growth and body composition is further evaluated by BIA.

15. The method according to claim 2, wherein the growth and body composition is further evaluated by DEXA.

16. The method according to claim 11, wherein the immunocompetence is evaluated by examination of the development of natural and acquired immunity.

17. The method according to claim 16, wherein the analyses include Fluorescent in Situ Hybridization of gut microbiota, and Enzyme-linked Immunospit assay of circulating immunoglobulin-secreting cells.

18. The method according to claim 2, wherein the correlating physiologic brain function comprises electrophysiologic/behavioral assessments.

19. The method according to claim 18, wherein the electrophysiologic/behavioral assessments comprise heart rate, respiratory rate, and EEG recordings in response to paradigms designed to measure social and startle response, language discrimination, and visual attention.

20. The method according to claim 2, wherein the cognitive/behavioral brain function is assessed by one or more standardized tests comprising the group of intelligence, achievement, language, temperament and behavior.

21. The method according to claim 2, wherein the correlating is for a period of 0-3 years.

22. The method according to claim 2, wherein the assessing of influence of family and home environment comprises assessing maternal intelligence and psychopathology, family socioeconomic status, stability, adaptability and cohesion.

23. The method according to claim 2, wherein the comparing comprises:
grasp force data;
finger-length data;
resting state recordings;
heart rate analyses;
respiratory rate; and
to skin conductance.

24. The method according to claim 23, wherein the grasp force compares left-right hand strength isometric force differences.

25. The method according to claim 23, wherein the finger length comprises ratios of second to fourth finger lengths.

26. The method according to claim 23, wherein the resting state is based on EEG, EMG and EOG data coupled with quantitative analyses of EEG, autonomic activity and video recordings.

27. The method according to claim 23, wherein the heart rate analyses comprises measuring heart beat-to-beat intervals in resting periods and in association with stimuli.

28. A method to determine a genetic-dietary link of blood group phenotypes and monosaccharide deficiencies and premature birth, said method comprising:
determining mothers' blood type, and
comparing said blood type to blood type of mothers having monosaccharide deficiencies and premature birth whose infants are enrolled in longitudinal evaluations.

29. The method according to claim 28, wherein the blood-group phenotypes comprise ABO and Lewis b phenotypes.

30. The method according to claim 29, wherein the determination further comprises saliva tests for determination of Lewis secretor status.

31. A composition comprising a custom blend of critical monosaccharides in a formulation to supplement infant diets deficient in said monosaccharides, comprising:
adding critical monosaccarides to mothers' milk deficient in said monosaccharides and/or common commercial term and preterm formulas devoid of said monosaccharides in order to simulate their composition in normal term mothers' milk.

32. The composition according to claim 31, wherein the critical monossacharides comprise one or more members of the group comprising fucose, mannose, glutamic acid and sialic acid.

33. The composition according to claim 32, wherein the sialic acid is endogenous sialic acid produced from glucosamine by an epimerase enzyme.

34. The composition according to claim 32, wherein the critical monossacharides are pharmaceutically compounded in a powder form.

35. The composition according to claim 32, wherein the critical monossacharides are compounded in a pharmaceutically acceptable carrier for intravenous administration.

36. The composition according to claim 32, wherein the critical monossacharides are compounded in a pharmaceutically acceptable carrier for enteral feeding.

37. A method for determining a probability of adverse reproductive events in a woman of reproductive age, said method comprising:
determining the presence in a biological sample from said woman of one or more biomarker,
wherein said biomarker correlates with a known risk of adverse reproductive events.

38. The method according to claim 37, wherein the woman is in gestation.

39. The method according to claim 37, wherein said sample is selected from the group comprising a blood sample, a serum sample, a plasma sample, milk sample, saliva sample and fetal cord blood sample.

40. The method according to claim 37, wherein said biomarker is selected from the group comprising fucose, mannose, glutamic acid and sialic acid.

41. The method according to claim 37, wherein said biomarker is further selected from the group comprising ABO blood phenotypes and Lewis phenotypes.

42. The method according to claim 37, wherein said adverse reproductive events comprise premature birth and glyconutrient deficiencies leading to compromised growth, health, physiologic and cognitive/behavioral brain function in premature and term infants during early childhood development.

43. A method of predicting occurrence of adverse reproductive events, the method comprising:
obtaining a test sample from a subject;
analyzing the obtained test sample for
ABO phenotype or Lewis phenotype biomarkers, and
one or more additional biomarkers that are indicative of the probability of adverse reproductive events; and
correlating the presence or amount of said biomarkers, with a biomarker burden and clinicopathological data of adversely affected infants, in order to deduce a probability of having adverse reproductive events.

44. The method of claim 43, wherein said additional biomarkers are selected from one or more of the group consisting of fucose, mannose, glutamic acid and sialic acid.

45. The method according to claim 43, wherein the correlating step is made in accordance with an algorithm drawn from the group consisting essentially of: linear or nonlinear regression algorithms; linear or nonlinear classification algorithms; ANOVA; neural network algorithms; and genetic algorithms.

46. The method according to claim 43, where the algorithm is a plurality of algorithms.

47. The method according to claim 46, wherein a tree algorithm, such as CART, MARS, or others, is trained to reproduce the performance of another machine-learning classifier or regressor.

48. The method according to claim 43, wherein said correlating step comprises:
determining level(s) of one or more biomarker(s) from a reproductive age or gestating woman;
comparing said levels values to women known to have had adverse reproductive events; and
training an algorithm to identify different patterns in said women that correlate with the presence or absence of said matched type of adverse reproductive events.

49. The method according to claim 47, wherein the training of said algorithm on characteristic biomarkers comprises the steps of:
obtaining numerous examples of
said biomarker data, and
historical clinical results corresponding to this biomarker data;
constructing an algorithm wherein
said biomarker levels and values are inputs to the algorithm, and
the historical clinical results are outputs of the algorithm; and
exercising the constructed algorithm to correlate said biomarker levels and values as inputs to the historical clinical results as outputs.

50. A kit useful for screening for monossacharide deficiencies and treatment in premature and term infants, comprising devices and reagents and an algorithm residing in a computer for measuring one or more of a group of biomarkers of a reproductive age woman and correlating with known adverse reproductive events.

51. The kit in accordance with claim 50, wherein said adverse reproductive events comprise premature birth, glyconutrient deficiencies leading to compromised growth, health, physiologic and cognitive/behavioral brain function in premature and term infants during early childhood development.

52. The kit in accordance with claim 50, wherein said group of biomarkers are comprised of a panel of fucose, mannose, glutamic acid, sialic acid, and ABO and Lewis phenotype markers.

53. A kit comprising devices and reagents and a computer algorithm installed on a computer for measuring one or more biomarkers in a biological sample from a woman of reproductive age and determining the probability in that woman for risk of adverse reproductive events by using the computer algorithm to correlate levels of said biomarkers,
wherein in response to the measured biomarkers, the algorithm determines for that woman a predictive value for adverse reproductive events and for treatment outcome.
